(19) United States
(12) Patent Application Publication
(10) Pub. No.: US 2002/0040010 A1
(43) Pub. Date: Apr. 4, 2002

(54) Use of agents to treat heart disorders

(76) Inventors: Anthony Rosenzweig, Newton, MA (US); Roger J. Hajjar, Cambridge, MA (US); Luis Guerrero, Norton, MA (US)

Correspondence Address: LOUIS MYERS, Fish & Richardson P.C., 225 Franklin Street, Boston, MA, 02110-2804, US

(21) Appl. No.: 09/789,894

(22) Filed: Feb. 21, 2001

Related U.S. Application Data
(63) Continuation-in-part of application No. 09/119,092, filed on Jul. 20, 1998, now abandoned.
(60) Provisional application No. 60/053,356, filed on Jul. 22, 1997.

(51) Int. Cl. A01K 67/00; A61K 48/00

(52) U.S. Cl. 514/44; 424/93.21; 800/3

(57) ABSTRACT

The invention provides methods of treating a subject at risk for a heart disorder. The method includes delivering a compound to the heart of a subject which alters the ratio of SERCA2a and phospholamban in the heart or heart cells of a subject. The invention also provides methods of evaluating a treatment for a heart disorder; a heart cell, into which has been introduced by somatic gene transfer, a nucleic acid which results in the expression of phospholamban; a heart tissue, into which has been introduced by somatic gene transfer, a nucleic acid which results in the expression of phospholamban; and a heart, into which has been introduced by somatic gene transfer, a nucleic acid which results in the expression of phospholamban.

### RELATED APPLICATIONS

This application is a continuation-in-part of U.S. patent application Ser. No. 09/119,092, filed Jul. 20, 1998, which claims the benefit of a previously filed Provisional Application No. 60/053,356 filed Jul. 22, 1997, both of which are hereby incorporated by reference.

### BACKGROUND OF THE INVENTION

The sarcoplasmic reticulum (SR) is an internal membrane system, which plays a critical role in the regulation of cytosolic Ca²⁺ concentrations and thus, excitation-contraction coupling in muscle. Contraction is mediated through the release of Ca²⁺ from the SR, while relaxation involves the active re-uptake of Ca²⁺ into the SR lumen by a Ca²⁺-ATPase. In cardiac muscle, the SR Ca²⁺-ATPase activity (SERCA2a) is under reversible regulation by phospholamban.

Phospholamban is a small phosphoprotein, about 6,080 daltons in size, which is an integral element of the cardiac SR membrane. Phospholamban is phosphorylated in vivo in response to β-adrenergic agonist stimulation. In the dephosphorylated state, phospholamban inhibits SR Ca²⁺-ATPase activity by decreasing the affinity of the enzyme for Ca²⁺.

Heart failure is characterized by a number of abnormalities at the cellular level in the various steps of excitation-contraction coupling of the cardiac cells. One of the key abnormalities in both human and experimental heart failure is a defect in SR function which is associated with abnormal intracellular Ca²⁺ handling. Deficient SR Ca²⁺ uptake during relaxation has been identified in failing hearts from both humans and animal models and has been associated with a decrease in the activity of SR Ca²⁺-ATPase activity and altered Ca²⁺ kinetics.

### SUMMARY OF THE INVENTION

In one aspect, the invention features a method of treating a subject, e.g., by treating a heart cell of the subject. The subject is a human, or a non-human animal. The method includes introducing into a heart cell, e.g., in a heart tissue, or in a heart, in vitro or in vivo, a nucleic acid which results in the expression of SERCA2a.

In a preferred embodiment, treating the heart cell includes modulating the ratio of phospholamban to SERCA2a in the heart cell.

In a preferred embodiment, the subject, e.g., a human or a non-human animal, is at risk for, or has, a heart disorder, e.g., heart failure, ischemia, arrhythmia, myocardial infarction, congestive heart failure, transplant rejection, abnormal heart contractility, or abnormal Ca+2 metabolism.

In a preferred embodiment, the heart disorder is heart failure, ischemia, arrhythmia, myocardial infarction, congestive heart failure, transplant rejection, abnormal heart contractility, or abnormal Ca+2 metabolism.

In a preferred embodiment, the nucleic acid is introduced into the subject by somatic gene transfer, e.g., by catheter perfusion. In another preferred embodiment, the nucleic acid is introduced into the subject by somatic gene transfer and is not introduced into the germ line of the subject.

In a preferred embodiment, the subject is a human.

In a preferred embodiment, the nucleic acid is introduced in vitro.

In a preferred embodiment, the nucleic acid is introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca2+ metabolism, e.g., intracellular Ca2+ concentration, e.g., peak or resting (Ca2+). SR Ca2+ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, PI3 kinase, AKT kinase, sodium-calcium exchanger, calcium channel (L and T), calsequestrin or calreticulin. The evaluation can be performed before, after, or during the treatment.

In another aspect, the invention features a method of treating a subject, e.g., by treating a heart cell of the subject. The subject is a human, or a non-human animal. The method includes introducing into the subject a nucleic acid which results in the expression of an antisense nucleic acid which is at least partially complementary to a phospholamban DNA sequence.

In a preferred embodiment, the subject is at risk for, or has, a heart disorder, e.g., heart failure, ischemia, arrhythmia, myocardial infarction, congestive heart failure, transplant rejection, abnormal heart contractility, or abnormal Ca+2 metabolism.

In a preferred embodiment, the heart disorder is heart failure, ischemia, arrhythmia, myocardial infarction, congestive heart failure, transplant rejection, abnormal heart contractility, or abnormal Ca+2 metabolism.

In a preferred embodiment, the nucleic acid is introduced into the subject by somatic gene transfer, e.g., by catheter perfusion. In another preferred embodiment, the nucleic acid is introduced into the subject by somatic gene transfer and is not introduced into the germ line of the subject.

In a preferred embodiment, the subject is a human, e.g., a human who is at risk for, or has, heart failure.

In a preferred embodiment, the nucleic acid is introduced in vitro.

In a preferred embodiment, the nucleic acid is introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca2+ metabolism, e.g., intracellular Ca2+ concentration, e.g., peak or resting (Ca2+). SR Ca2+ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, PI3 kinase, AKT kinase, sodium-calcium exchanger, calcium channel (L and T), calsequestrin or calreticulin. The evaluation can be performed before, after, or during the treatment.

In another aspect, the invention features a method of treating a subject, e.g., by treating a heart cell of the subject. The subject is a human, or a non-human animal. The method includes introducing into the subject, e.g., the heart of the subject, a first nucleic acid which results in the expression of an antisense nucleic acid which is at least partially complementary to a phospholamban DNA sequence, and introducing into the subject a second nucleic acid which results in the expression of SERCA2.

In a preferred embodiment, the subject, e.g., a human or a non-human animal, is at risk for, or has, a heart disorder, e.g., heart failure, ischemia, arrhythmia, myocardial infarction, congestive heart failure, transplant rejection, abnormal heart contractility, or abnormal Ca+2 metabolism.

In a preferred embodiment, the heart disorder is heart failure, ischemia, arrhythmia, myocardial infarction, congestive heart failure, transplant rejection, abnormal heart contractility, or abnormal Ca+2 metabolism.

In a preferred embodiment, the first and second nucleic acids are introduced into the subject by somatic gene transfer, e.g., by catheter perfusion. In another preferred embodiment, the nucleic acids are introduced into the subject by somatic gene transfer and are not introduced into the germ line of the subject.

In a preferred embodiment, the subject is a human.

In a preferred embodiment, the nucleic acids are introduced in vitro.

In a preferred embodiment, the nucleic acids re introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca2+ metabolism, e.g., intracellular Ca2+ concentration, e.g., peak or resting (Ca2+). SR Ca2+ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, PI3 kinase, AKT kinase, sodium-calcium exchanger, calcium channel (L and T), calsequestrin or calreticulin. The evaluation can be performed before, after, or during the treatment.

In another aspect, the invention features, a method of evaluating a treatment for a heart disorder. The method includes:providing a heart cell, into which has been introduced by somatic gene transfer, a nucleic acid which results in the expression of phospholamban; administering the treatment to the heart cell; and evaluating the effect of the treatment on the heart cell, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the cell is derived from an experimental animal or a human. In preferred embodiments the cell can be cultured and/or immortalized.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments, the nucleic acid is introduced into the heart cell by way of a vector suitable for somatic gene transfer, e.g., a viral vector, e.g., an adenoviral vector.

In another aspect, the invention features, a method of evaluating a treatment for a heart disorder. The method includes: providing a heart, into some or all the cells of which has been introduced, by somatic gene transfer, a nucleic acid which results in the expression of phospholamban; administering the treatment to the heart; and evaluating the effect of the treatment on the heart, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the heart is derived from an experimental animal or a human.

In preferred embodiments, the nucleic acid encodes a phospholamban protein. The phospholamban can be from the same species that the heart is from or it can be from a different species. For example, a mouse phospholamban can be expressed in a mouse heart or a human phospholamban can be expressed in the heart of an experimental animal. The phospholamban can be delivered to the heart using methods described herein.

In preferred embodiments, the nucleic acid is introduced into the heart by way of a vector suitable for somatic gene transfer, e.g., a viral vector, e.g., an adenoviral vector.

In another aspect, the invention features, a method of evaluating a treatment for a heart disorder. The method includes: providing heart tissue into some or all of the cells of which has been introduced, by somatic gene transfer, a nucleic acid which results in the expression of phospholamban; administering the treatment to the heart tissue; and evaluating the effect of the treatment on the heart tissue, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the heart tissue is derived from an experimental animal or a human.

In preferred embodiments, the nucleic acid encodes a phospholamban protein. The phospholamban can be from the same species that the heart tissue is from or it can be from a different species. For example, a mouse phospholamban can be expressed in a mouse heart tissue or a human phospholamban can be expressed in heart tissue from an experimental animal.

In preferred embodiments, the nucleic acid is introduced into the heart tissue by way of a vector suitable for somatic gene transfer, e.g., a viral vector, e.g., an adenoviral vector.

In another aspect, the invention features, a method of evaluating a treatment for a heart disorder. The method includes: providing a first and a second heart cell, into each of which has been introduced, by somatic gene transfer, a nucleic acid which results in the expression of phospholamban; administering the treatment to a first heart cell, preferably in vitro; evaluating the effect of the treatment on the first heart cell; administering the treatment to a second heart cell, preferably in vivo; and evaluating the effect of the treatment on the second heart cell, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments, the first and second cell can be from the same or different animals, can be from the same or different species, e.g., the first cell can be from a mouse and the second cell can be from a human or both cells can be human. The first and second cell can have the same or different genotypes. In further preferred embodiments the evaluation of the treatment in the first cell can be the same or different from the evaluation of the treatment in the second cell, e.g., the intracellular Ca²⁺ concentration can be measured in the first cell and the SR Ca²⁺-ATPase activity can be measured in the second cell or the intracellular Ca²⁺ concentration can be measured in both cells.

In another aspect, the invention features, a method of evaluating a treatment for a heart disorder. The method includes: providing a first administration of a treatment to a heart cell, into which has been introduced by somatic gene transfer, a nucleic acid which results in the expression of phospholamban; evaluating the effect of the first administration on the heart cell; providing a second administration of a treatment to a heart cell, into which has been introduced by somatic gene transfer, a nucleic acid which results in the expression of phospholamban; and evaluating the effect of the second administration on the heart cell, thereby evaluating a treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments the first and second administration can be administered to the same or to different cells. The first and second administration can be administered under the same or different conditions, e.g., the first administration can consist of a relatively low level treatment, e.g., a lower concentration of a substance, and the second administration can consist of a relatively high level treatment, e.g., a higher concentration of a substance or both administrations can consist of the same level treatment.

In another aspect, the invention features, a method of evaluating a treatment for a heart disorder. The method includes: providing a heart cell, into which has been introduced by somatic gene transfer, a nucleic acid which results in the expression of phospholamban; administering the treatment to the heart cell; evaluating the effect of the treatment on the heart cell; providing a heart, into which has been introduced by somatic gene transfer, a nucleic acid which results in the expression of phospholamban; administering the treatment to the heart ; and evaluating the effect of the treatment on the heart, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell and/or the heart is from or it can be from a different species. For example, a mouse phospholamban can be expressed in a mouse heart cell and/or heart or a human phospholamban can be expressed in a heart cell and/or heart from an experimental animal.

In preferred embodiments the treatment can be administered to the heart cell in vitro and to the heart in vivo or the treatment can be administered to the heart cell and to the heart in vitro.

In another aspect, the invention features, a method of delivering a compound to the heart of a subject. The method includes: restricting the aortic flow of blood out of the heart, such that blood flow is re-directed to the coronary arteries; introducing the compound into the lumen of the circulatory system such that it flows into the coronary arteries; allowing the heart to pump while the aortic outflow of blood is restricted, thereby allowing the compound to flow into and be delivered to the heart; and reestablishing the flow of blood to the heart.

In preferred embodiments, the compound includes: a nucleic acid which directs the expression of a peptide, e.g., a phospholamban or a SR Ca²⁺-ATPase and a viral vector suitable for somatic gene delivery, e.g., an adenoviral vector.

In preferred embodiments, the subject is at risk for a heart disorder, e.g., heart failure, ischemia, arrhythmia, myocardial infarction, congestive heart failure, transplant rejection.

In preferred embodiments, the subject can be a human or an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a disorder, e.g., a heart disorder.

In preferred embodiments, the method further includes restricting blood flow into the left side of the heart, e.g., by restricting the pulmonary circulation through obstraction of the pulmonary artery, so as to lessen dilution of the compound.

In preferred embodiments the method further includes opening the pericardium and introducing the compound, e.g., using a catheter.

In preferred embodiments, the compound is: introduced into the lumen of the aorta, e.g., the aortic root, introduced into the coronary ostia or introduced into the lumen of the heart.

In preferred embodiments, the nucleic acid, which directs the expression of the peptide, is homogeneously overexpressed in the heart of the subject.

In another aspect, the invention features, a heart cell, into which has been introduced by somatic gene transfer, a nucleic acid which results in the expression of phospholamban. The heart cell can be provided as a purified preparation.

In another aspect, the invention features, a heart tissue, into which has been introduced by somatic gene transfer, a nucleic acid which results in the expression of phospholamban. The heart tissue can be provided as a tissue preparation.

In another aspect, the invention features, a heart, into which has been introduced by somatic gene transfer, a nucleic acid which results in the expression of phospholamban. The heart can be provided in a subject or ex vivo, i.e. removed from a subject.

In another aspect, the invention features, a method for treating a subject at risk for a heart disorder. The method includes introducing into somatic heart tissue of the subject, a nucleic acid which encodes phospholamban.

In preferred embodiments, the nucleic acid is introduced using the methods described herein.

In preferred embodiments, the phospholamban can be from the same species as the subject or it can be from a different species. For example, a human phospholamban can be introduced into a human heart or a human phospholamban can be can be introduced into the heart of an experimental animal.

In preferred embodiments, the nucleic acid is introduced into the heart by way of a vector suitable for somatic gene transfer, e.g., a viral vector, e.g., an adenoviral vector.

In preferred embodiments, the subject can be a human, an experimental animal, e.g., a rat or a mouse, a domestic animal, e.g., a dog, cow, sheep, pig or horse, or a non-human primate, e.g., a monkey. The subject can be suffering from a cardiac disorder, such as heart failure, ischemia, myocardial infarction, congestive heart failure, arrhythmia, transplant rejection and the like.

As used herein, the term “treatment” refers to a procedure (e.g., a surgical method) or the administration of a substance, e.g., a compound which is being evaluated for use in the alleviation or prevention of a heart disorder or symptoms thereof. For example, such treatment can be a surgical procedure, or the administration of a therapeutic agent such as a drug, a peptide, an antibody, an ionophore and the like.

As used herein, the term “heart disorder” refers to a structural or functional abnormality of the heart, that impairs its normal functioning. For example, the heart disorder can be heart failure, ischemia, myocardial infarction, congestive heart failure, arrhythmia, transplant rejection and the like. The term includes disorders characterized by abnormalities of contraction, abnormalities in Ca²⁺ metabolism, and disorders characterized by arrhytmia.

As used herein, the term “heart cell” refers to a cell which can be: (a) part of a heart present in a subject, (b) part of a heart which is maintained in vitro, (c) part of a heart tissue, or (d) a cell which is isolated from the heart of a subject. For example, the cell can be a cardiac myocyte.

As used herein, the term “heart” refers to a heart present in a subject or to a heart which is maintained outside a subject.

As used herein, the term “heart tissue” refers to tissue which is derived from the heart of a subject.

As used herein, the term “somatic gene transfer” refers to the transfer of genes into a somatic cell as opposed to transferring genes into the germ line.

As used herein, the term “compound” refers to a compound, which can be delivered effectively to the heart of a subject using the methods of the invention. Such compounds can include, for example, a gene, a drug, an antibiotic, an enzyme, a chemical compound, a mixture of chemical compounds or a biological macromolecule.

As used herein, the term “subject” refers to an experimental animal, e.g., a rat or a mouse, a domestic animal, e.g., a dog, cow, sheep, pig or horse, a non-human primate, e.g., a monkey and in the case of therapeutic methods, humans. However, it is noted that human cells, tissue or hearts can be used in vitro evaluations. A subject can suffer from a heart disorder, such as heart failure, ischemia, myocardial infarction, congestive heart failure, arrhythmia, transplant rejection and the like. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, PI₃ kinase, AKT kinase or members of the caspase family of proteins. Calcium regulatory proteins include phospholamban, SR Ca2⁺ ATPase, sodium-calcium exchanger, calcium channel (L and T), calsequestrin or calreticulin. The experimental animal can be an animal model for a heart disorder, such as a hypertensive mouse or rat.

As used herein, the term “misexpression” refers to a non-wild type pattern of gene expression. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. Misexpression includes any expression from a transgenic nucleic acid.

As used herein, the term “restricting the aortic flow of blood out of the heart” refers to substantially blocking the flow of blood into the distal aorta and its branches. For example, at least 50% of the blood flowing out of the heart is restricted, preferably 75% and more preferably 80, 90, or 100% of the blood is restricted from flowing out of the heart. The blood flow can be restricted by obstructing the aorta and the pulmonary artery, e.g., with clamps.

As used, herein, the term “introducing” refers to a process by which a compound can be placed into a chamber or the lumen of the heart of a subject. For example, the pericardium can be opened and the compound can be injected into the heart, e.g., using a syringe and a catheter. The compound can be: introduced into the lumen of the aorta, e.g., the aortic root, introduced into the coronary ostia or introduced into the lumen of the heart.

As used herein, the terms “homogeneous fashion” and “homogeneously overexpressing” are satisfied if one or more of the following requirements are met: (a) the compound contacts at least 10%, preferably 20, 20, 40, 50, 60, 70, 80, 90 or 100% of the cells of the heart and (b) at least 10%, preferably 20, 20, 40, 50, 60, 70, 80, 90 or 100% of the heart cells take up the compound.

As used herein, the term “purified preparation” refers to a preparation in which at least 50, preferably 60, 70, 80, 90 or 100% of the cells are heart cells into which phospholamban has been introduced by somatic gene transfer.

The methods of the invention allow rapid and low cost development of cardiac overexpression models. The methods of the invention also provide ways of examining multiple genes interacting in transgenic models, testing gene therapy approaches and evaluating treatments of cardiac disorders.

Other features and advantages of the invention will be apparent from the following detailed description, and from the claims.

### DETAILED DESCRIPTION

### BRIEF DESCRIPTION OF THE DRAWINGS

The drawings are first briefly described.

FIG. 1 is a graph depicting protein levels of SR Ca²⁺-ATPase in uninfected cardiomyocytes (n=8) and in cardiomyocytes infected for 48 hours with 1, 10, and 100 pfu/cell of Ad.RSV.PL.

FIG. 2 is a graph depicting protein levels of phospholamban and SERCA2a in uninfected cardiomyocytes (n=8) and in cardiomyocytes infected for 48 hours with 10 pfu/cell with either Ad.RSV.PL and/or Ad.RSV.SERCA2a. There were no significant differences between the phospholamban protein levels in the group of myocytes infected with Ad.RSV.PL alone at a multiplicity of infection of 10 pfu/cell and the group of myocytes infected with Ad.RSV.PL at a multiplicity of infection of 10 pfu/cell and Ad.RSV.SERCA2a at a multiplicity of infection of 10 pfu/cell (P>2). Similarly, there were no significant differences between the SERCA2a protein levels in the group of myocytes infected with Ad.RSV.SERCA2a alone at a multiplicity of infection of 10 pfu/cell and the group of myocytes infected with Ad.RSV.PL at a multiplicity of infection of 10 pfu/cell and Ad.RSV.SERCA2a at a multiplicity of infection of 10 pfu/cell (P>2).

FIG. 3A is a graph depicting SERCA2a activity as a function of Ca²⁺ in membrane preparations of uninfected cardiomyocytes (▪, n=6), cardiomyocytes infected with 10 pfu/cell of Ad.RSV.PL (●, n=6), and cardiomyocytes infected with 10 pfu/cell of Ad.RSV.PL and 10 pfu/cell of Ad.RSV.SERCA2a (▴, n=6).

FIG. 3B is a graph depicting the effect of increasing concentrations of cyclopiazonic acid (CPA) on SRECA2 activity at a [Ca²⁺] of 10 μmol/L in membrane preparations of uninfected cardiomyocytes (▪, n=6), cardiomyocytes infected with 10 pfu/cell of Ad.RSV.PL (●, n=6), and cardiomyocytes infected with 10 pfu/cell of Ad.RSV.PL and 10 pfu/cell of Ad.RSV.SERCA2a (▴, n=6).

FIG. 4A shows intracellular Ca²⁺ transients and shortening in an uninfected cardiomyocyte and in a cardiomyocyte infected for 48 hours with 10 pfu/cell of Ad.RSV. βgal and stimulated at 1 Hz.

FIG. 4B shows intracellular Ca²⁺ transients and shortening in an uninfected cardiomyocyte and in a cardiomyocyte infected for 48 hours with 1, 10, and 100 pfu/cell of Ad.RSV.PL stimulated at 1 Hz.

FIG. 4C shows intracellular Ca²⁺ transients and shortening in an uninfected cardiomyocyte, a cardiomyocyte infected with 1-pfu/cell of Ad.RSV.PL, and a cardiomyocyte infected with 10 pfu/cell of Ad.RSV.PL and 10 pfu/cell of Ad.RSV.SERCA2a for 48 hours, stimulated at 1 Hz.

FIG. 5A is a graph showing the mean of the peak of the intracellular Ca²⁺ transients in uninfected cardiomyocytes (n=10), cardiomyocytes infected with 10 pfu/cell of Ad.RSV.PL (n=12), and cardiomyocytes infected with 10 pfu/cell of Ad.RSV.PL and 10 pfu/cell of Ad.RSV.SERCA2a (n=10) for 48 hours and stimulated at 1 Hz.

FIG. 5B is a graph showing the mean of the resting levels of [Ca²⁺] in uninfected cardiomyocytes (n=10), cardiomyocytes infected with 10 pfu/cell of ad.RSV.PL (n=12), and cardiomyocytes infected with 10 pfu/cell of Ad.RSV.PL and 10 pfu/cell of Ad.RSV.SERCA2a (n=10) for 48 hours, stimulated at 1 Hz.

FIG. 5C is a graph showing the mean of the time to 80% relaxation of the intracellular Ca²⁺ transients in uninfected cardiomyocytes (n=10), cardiomyocytes infected with 10 pfu/cell of Ad.RSV.PL (n=12), and cardiomyocytes infected with 10 pfu/cell of Ad.RSV.PL and 10 pfu/cell of Ad.RSV.SERCA2a (n=10) for 48 hours, stimulated at 1 Hz. P<0.05 compared with uninfected cells. P<0.05 compared with Ad.RSV.PL (multiplicity of infection of 10 pfu/cell).

FIG. 6 is a graph showing the effect of increasing concentrations of Isoprotenerol on the time course of the intracellular Ca²⁻ transients in uninfected cardiomyocytes (n=5) and cardiomyocytes infected with 10 pfu/cell of Ad.RSV.PL (n=5), stimulated at 1 Hz.

FIG. 7A is a graph showing the response of intracellular Ca²⁺ transients to increasing frequency of stimulation in an uninfected cardiomyocyte.

FIG. 7B is a graph showing the response of intracellular Ca²⁺ transients to increasing frequency of stimulation in a cardiomyocyte infected with 10 pfu/cell of Ad.RSV.PL.

FIG. 7C is a graph showing the response of intracellular Ca²⁺ transients to increasing frequency of stimulation in a cardiomyocyte infected with 10 pfu/cell of Ad.RSV.PL and 10 pfu/cell of Ad.RSV.SERCA2a for 48 hours.

FIG. 8 shows intracavitary pressure tracings from rats 48 hours after cardiac gene transfer with either Ad.EGFP (left) or Ad.PL (right). The pressure tracing of the Ad.PL transduced hearts displays a markedly prolonged relaxation and reduced pressure development.

FIG. 9 is a drawing showing the somatic gene delivery method.

FIG. 10A is a graph demonstrating that infection of neonatal cardiac myocytes with the construct Ad.asPL increased the contraction amplitude and significantly shortened the time course of the contraction.

FIG. 10B is a graph demonstrating that adenovirus-mediated gene transfer of the antisense cDNA for phospholamban results in a modification of intracellular calcium handling.

FIG. 11 is a graph of survival curves for sham operated animals, and failing animals expressing Sarcoplasmic Reticulum Calcium ATPase through gene transfer. Sham, n=14; sham+Ad.bgal-GFP, n=12; sham+Ad.SERCA2a, n=14; failing, n=14; failing+Ad.bgal-GFP, n=12; failing+SERCA2a, n=16

FIG. 12 is a bar graph of ATPase activity measured vs [Ca²⁺] in membrane preparations from sham rats infected with Ad.bgal-GFP (n=4), preparations from failing rat hearts infected with Ad.bgal-GFP (n=4) and preparations of failing hearts infected with Ad.SERCA2a (n=4).

FIG. 13. The failing spectrum illustrates that the PCr-to-ATP ratio and the PCr and ATP contents in the failing heart are lower than in the nonfailing sham heart. In the spectrum of the failing+Ad.SERCA2a heart, the PCr-to-ATP ratio is restored towards normal.

FIG. 14. Left ventricular volumes measured using piezoelectric crystals placed on the surface of the left ventricle in open chested animals. Note the increase in left ventricular volume in failing hearts which is restored towards normal following gene transfer of SERCA2a.

Phospholamban and Heart Disorder

The inventors have discovered that somatic gene transfer, e.g., adenoviral gene transfer, is particularly effective in mammalian myocardium both in vivo and in vitro and particularly useful for screening treatments suitable for heart disorders. Adenoviral gene transfer of SERCA2a is both dose dependent and time dependent in rat neonatal cardiomyocytes. An adenovirus encoding phospholamban under the RSV promoter, provided a 4-fold increase in phospholamban, which was also dose dependent. The smaller size of phospholamban compared with SERCA2a (6 kD in its monomer form compared with 110 kD) may explain, at least in part, the more effective protein expression by Ad.RSV.PL than by Ad.RSV.SERCA2a under similar conditions. Nevertheless, using these recombinant adenoviruses, significant overexpression of phospholamban and SERCA2a was achieved, individually and in combination. Co-infection with both Ad.RSV.PL and Ad.RSV.SERCA2a mediated overexpression of both SERCA2a and phospholamban that was the same as the expression from infection with either Ad.RSV.PL or Ad.RSV.SERCA2a alone. The ability to simultaneously manipulate expression of multiple proteins in the context of primary myocytes is an advantage of somatic gene transfer for the study of interacting components of complex systems.

The expression of phospholamban relative to SERCA2a is altered in a number of disease states. In hypothyroidism phospholamban levels are increased, whereas in hyperthyroidism phospholamban levels are decreased. An increased ratio of phospholamban to SERCA2a is an important characteristic of both human and experimental heart failure. Both experimental and human heart failure are characterized by a prolonged Ca²⁺ transient and impaired relaxation. Increasing levels of phospholamban relative to SERCA2a significantly altered intracellular Ca²⁺ handling in the isolated cardiomyocytes by prolonging the relaxation phase of the Ca²⁺ transient, decreasing Ca²⁺ release, and increasing resting Ca²⁺. These results show that altering the relative ratio of phospholamban to SERVA2a can account for the abnormalities in Ca²⁺ handling observed in failing ventricular myocardium. In addition, overexpressing SERCA2a can largely “rescue” the phenotype created by increasing the phospholamban-to-SERCA2a ratio. Restoring the normal phospholamban-to-SERCA2a ratio through somatic gene transfer can correct the abnormalities of Ca²⁺ handling and contraction seen in failing hearts.

Evaluation of Treatment

A treatment can be evaluated by assessing the effect of the treatment on a parameter related to contractility. For example, SR Ca²⁺ ATPase activity or intracellular Ca²⁺ concentration can be measured, using the methods described above. Furthermore, force generation by hearts or heart tissue can be measured using methods described in Strauss et al., Am. J. Physiol., 262:1437-45, 1992, the contents of which are incorporated herein by reference.

In many drug screening programs which test libraries of therapeutic agents and natural extracts, high throughput assays are desirable in order to maximize the number of therapeutic agents surveyed in a given period of time. Assays which are performed in cell-free systems, such as may be derived with cardiac muscle cell extracts, are preferred as “primary” screens in that they can be generated to permit rapid development and relatively easy detection of the parameter being measured, e.g., the intracellular levels of Ca²⁺, which is mediated by a test therapeutic agent. Moreover, the effects of cellular toxicity and/or bioavailability of the test therapeutic agent can be generally ignored in the in vitro system, the assay instead being focused primarily on the effect of the therapeutic agent on the parameter being measured, e.g., the intracellular levels of Ca²⁺. It is often desirable to screen candidate treatments in two stages, wherein the first stage is performed in vitro, and the second stage is performed in vivo.

The efficacy of a test therapeutic agent can be assessed by generating dose response curves from data obtained using various concentrations of the test therapeutic agent. Moreover, a control assay can also be performed to provide a baseline for comparison. In the control assay, the heart cell is incubated in the absence of a test agent.

Propagation of Heart Cells

A heart cell culture can be obtained by allowing heart cells to migrate out of fragments of heart tissue adhering to a suitable substrate (e.g., a culture dish) or by disaggregating the tissue, e.g., mechanically or enzymatically to produce a suspension of heart cells. For example, the enzymes trypsin, collagenase, elastase, hyaluronidase, DNase, pronase, dispase, or various combinations thereof can be used. Trypsin and pronase give the most complete disaggregation but may damage the cells. Collagenase and dispase give a less complete dissagregation but are less harmful. Methods for isolating tissue (e.g., heart tissue) and the disaggregation of tissue to obtain cells (e.g., heart cells) are described in Freshney R. I., Culture of Animal Cells, A Manual of Basic Technique, Third Edition, 1994, the contents of which are incorporated herein by reference.

Viral Vectors Suitable for Somatic Gene Transfer

Expression vectors, suitable for somatic gene transfer, can be used to express the compound, e.g., the phospholamban gene. Examples of such vectors include replication defective retroviral vectors, adenoviral vectors and adeno-associated viral vectors. Adenoviral vectors suitable for use by the methods of the invention include (Ad.RSV.lacZ), which includes the Rous sarcoma virus promoter and the lacZ reporter gene as well as (Ad.CMV.lacZ), which includes the cytomegalovirus promoter and the lacZ reporter gene. Methods for the preparation and use of viral vectors are described in WO 96/13597, WO 96/33281, WO 97/15679, and Trapnell et al., Curr. Opin. Biotechnol. 5(6):617-625, 1994, the contents of which are incorporated herein by reference.

Expression of Phospholamban

The nucleic acid which results in the overexpression of phospholamban can be derived from the natural phospholamban gene including all the introns and exons, it can be a cDNA molecule derived from the natural gene (Fujji et al., J. Biol. Chem. 266:11669-11675, 1991, the contents of which are incorporated herein by reference) or a chemically synthesized cDNA molecule. The nucleic acid encoding the phospholamban protein can be under the control of the naturally occurring promoter or any other promoter that drives a high level expression of the phospholamban gene.

The following examples which further illustrate the invention should not be construed as limiting.

### EXAMPLES

1. Construction of E1-deleted Recombinant Adenovirus Vectors

The construction of Ad.RSV.SERCA2a has been described in detail by Hajjar et al., Circulation, 95: 423-429, 1997, the contents of which are incorporated herein by reference. Ad.RSV.βgal, which carries a nuclear localizing form of β-galactosidase, is described in Dong et al., J. Biol. Chem. 27:29969-77, 1996, the contents of which are incorporated herein by reference. The rabbit phospholamban cDNA is described in Lylton J. MacLennan D. H., J. Biol. Chem., 1988, 263:15024-15031, the contents of which are incorporated herein by reference. Briefly, the phospholamban cDNA was subcloned into the bacterial plasmid vector pAdRSV4, which uses the RSV long terminal repeat as a promoter and the SV40 polyadenylation signal and contains map units with adenovirus sequences from 0 to 1 and from 9 to 16. The position and orientation of the phospholamban cDNA were confirmed by restriction enzyme digestion and by polymerase chain reaction. The plasmid vector containing phospholamban (pAd.RSV-PL) was then cotransfected into 293 cells with PJM17. The homologous recombinants between pAd.RSV.PL and pJM17 contain the phospholamban cDNA substituted for E1. By use of this strategy, independent plaques were isolated, and expression of phospholamban protein was verified by immunostaining. A positive plaque was further plaque-purified, and protein expression was reconfirmed to yield the recombinant adenovirus Ad.RSV.PL. This adenovirus is structurally similar to Ad.RSV.βgal and to Ad.RSV.SERCA2a, described in Dong O. et al., J. Biol. Chem, 1996, 271:29969-29977, 1976. The recombinant viruses were prepared as high-titer stocks by propagation in 293 cells as described in Graham, F. L. et al., Methods in Molecular Biology: Gene Transfer and Expression Protocols, 1991, 109-128, the contents of which are incorporated herein by reference. The titers of stocks used for these studies were as follows: 3.1×10¹⁴ pfu/mL for Ad.RSV.PL 2.6×10¹⁰ pfu/m: for Ad.RSV.SERCA2a, and 2.7×10¹⁴ pfu/mL for Ad.RSV. gal, with a particle-to-pfu ratio of 40:1, 42:1, and 37:1, respectively.

2. Preparation of Neonatal Cardiomyocytes

Spontaneously beating cardiomyocytes were prepared from 1 to 2 day old rats and cultured in P-10 medium (GIBCO,BRL) in the presence of 5% fetal calf serum and 10% horse serum for 3 days as described previously in Kang J. X. et al., Proc. Natl. Acad. Sci. U.S.A., 1995, 92:3097-4001 and Kang J. X. and Leaf A., Euro. J. Pharmacol., 1996, 297:97-106, the contents of which are incorporated herein by reference. Measurements of cell shortening and cytosolic Ca²⁺ were performed on neonatal cardiomyocytes cultured on round, coated, glass coverslips (0.1 mm thickness, 31 mm diameter) in 35 mm culture dishes. Cells were counted using a hemocytometer. Approximately 5×10⁵ cells were plated in each coverslip.

3. Adenoviral Infection of Isolated Cells

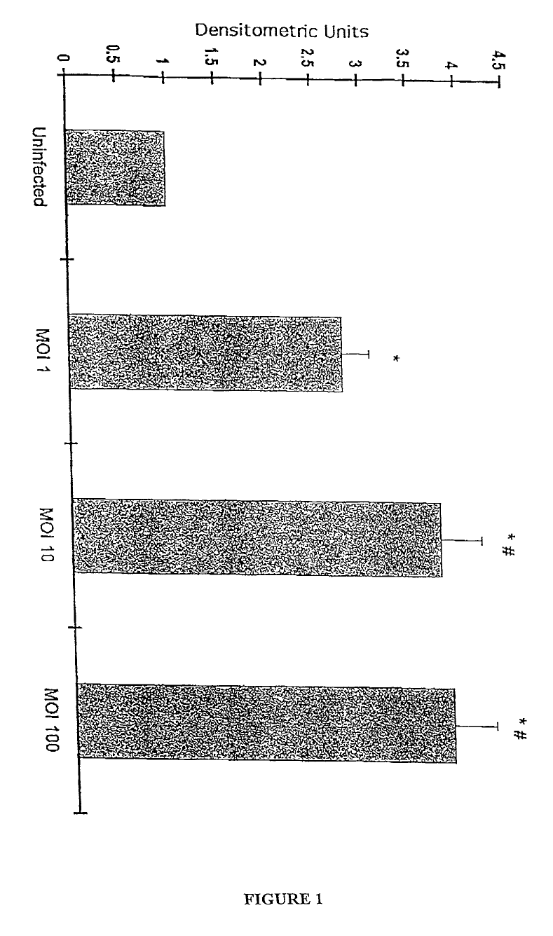

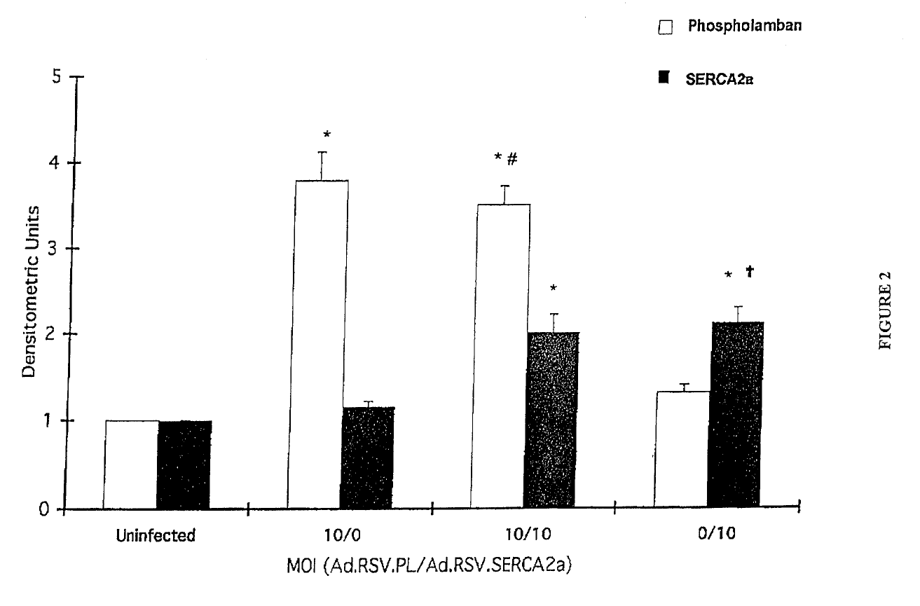

In three different infection experiments with increasing concentrations of Ad.RSV.βgal, the percentages of cells expressing βgal after 48 hours, by histochemical staining in 10 different high-power fields were 98.2% (multiplicity of infection, 1 pfu/cell), 99.1% (multiplicity of infection, 10 pfu/cell), and 100% (multiplicity of infection, 100 pfu/cell). In a similar manner, myocardial cells were infected with three concentrations of Ad.RSV.PL 1.0, 10. and 100 pfu/cell for 48 hours. Infection with either Ad.RSV.βgal, Ad.RSV.PL, or Ad.RSV.SERCA2a did not change the morphology of the cells. For each infection experiment with the adenovirus, one myocyte was used to measure functional parameters. As shown in FIG. 1, there was a 4-fold increase in phospholamban protein levels in a dose-dependent increase in the protein expression of phospholamban between 1 and 10 pfu/cell but no further increases between 10 and 100 pfu/cell. Coinfection of Ad.RSV.SERCA2a with Ad.RSV.PL produced an increase in protein expression of both SERCA2a and phospholamban, as shown by the immunoblot in FIG. 2. There were no significant differences between the phospholamban protein levels in the group of myocytes infected with Ad.RSV.PL alone at a multiplicity of infection of 10 pfu/cell and the group of myocytes infected with Ad.RSV.PL at an multiplicity of infection of 10 pfu/cell and Ad.RSV.SERCA2a at a multiplicity of infection of 10 pfu/cell (P>2). Similarly, there were no significant differences between the SERCA2a protein levels in the group of myocytes infected with Ad.RSV.SERCA2a alone at a multiplicity of infection of 10 pfu/cell and the group of myocytes infected with Ad.RSV.PL at a multiplicity of infection of 10 pfu/cell and Ad.RSV.SERCA2a at an multiplicity of infection of 10 pfu/cell (P>2).

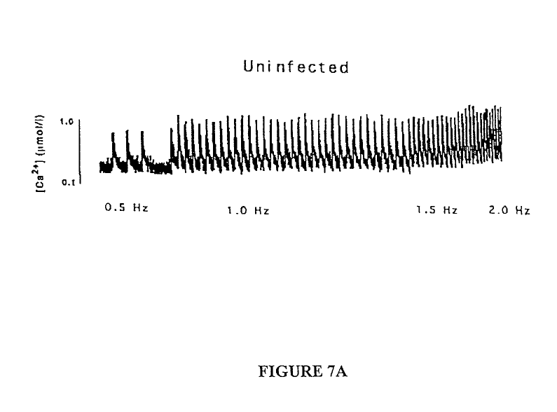

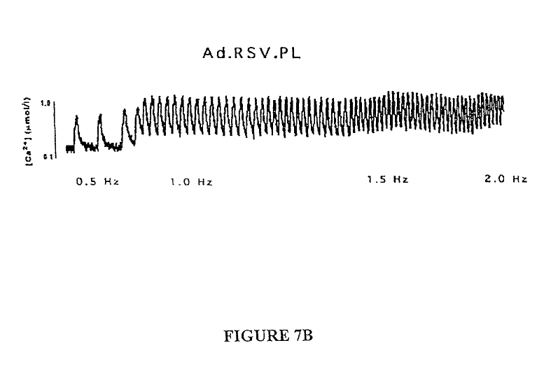

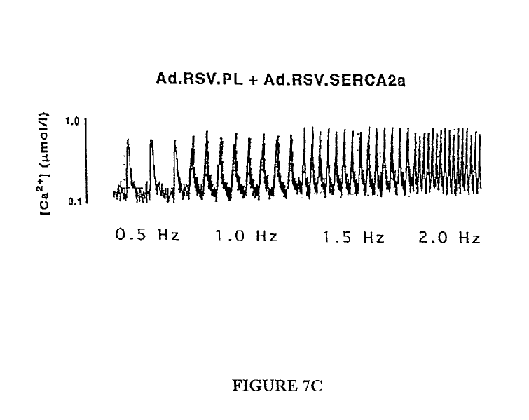

As shown in FIG. 7, cardiomyocytes infected with Ad.RSV.PL (multiplicity of infection of 10 pfu/cell) exhibited a significant increase in resting Ca²⁺ not evident in uninfected cells. Furthermore, coinfection with Ad.RSV.SERCA2a (multiplicity of infection of 10 pfu/cell) restored the frequency response to normal.

The response to increasing stimulation frequencies in mammalian cardiomyocytes is governed by the SR. We have shown that in the uninfected cardiomyocytes, an increase in stimulation frequency did not significantly alter either peak or resting (Ca²⁺). This response is typical of rat cardiomyocytes that have either a flat response to increasing frequency of stimulation or a decrease in contractile force. However, in cardiomyocytes infected with Ad.RSV.PL, there was a significantly greater increase in resting (Ca²⁺) and a decrease in peak (Ca²⁺). These results would suggest that diminished SR Ca²⁺ uptake leads to a diminished CA²⁺ release, which becomes even more accentuated at higher frequencies of stimulation.

3. Intracellular Ca²⁺ Measurements and Cell Shortening Detection

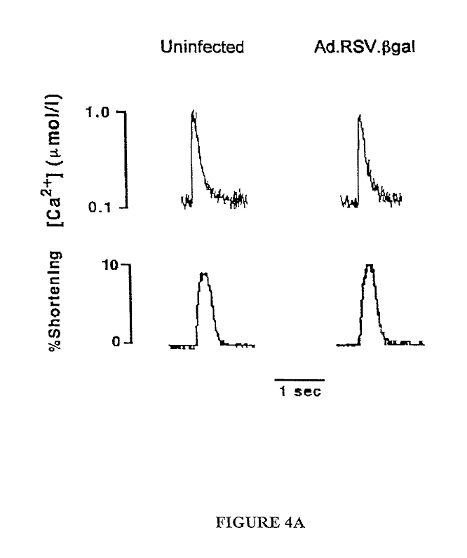

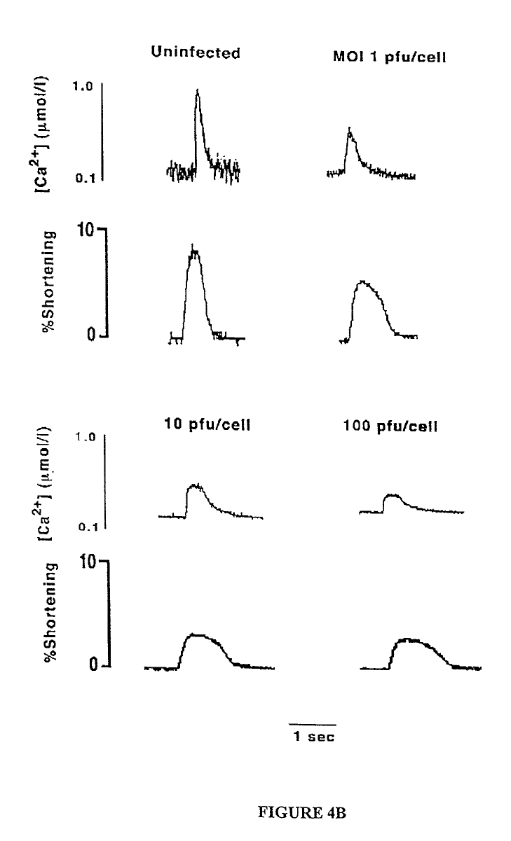

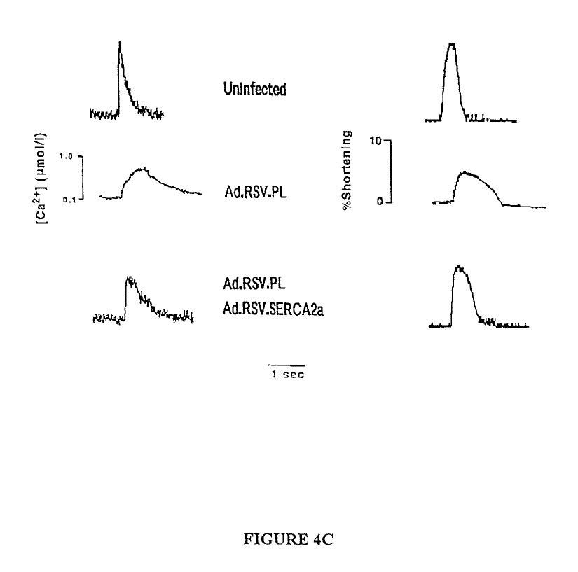

Measurements of intracellular Ca²⁺ and cell shortening were performed as described earlier in Hajjar et al. (1997), Kang et al. (1995) and Kang et al (1996), the contents of which are incorporated herein by reference. Briefly, myocardial cells were loaded with the Ca²⁺ indicator fura 2 by incubating the cells in medium containing 2 μmol/L fura 2-AM (Molecular Probex) for 30 minutes. The cells were then washed with PBS and allowed to equilibrate for 10 minutes in a light-sealed temperature-controlled chamber (32° C.) mounted on a Zeiss Axlovers 10 inverted microscope (Zeiss). The coverslip was superfused with a HEPES-buffered solution at a rate of 20 mL/h. Cells were stimulated at different frequencies (0.1 to 2.0 Hz) using an external stimulator (Grass Instruments). A dual excitation spectrofluorometer (IONOPTIX) was used to record fluorescence emissions (505 nm) elicited from exciting wavelengths of 360 and 380 nm. [Ca²⁺] was calculated according to the following formula: [Ca²⁺]=K_{d}(R−Rₘᵢₙ)/(Rₘₐₓ−R)D, where R is the ratio of fluorescence of the cell at 360 and 380 nm: Rₘᵢₙ and Rₘₐₓ represent the ratios of fura 2 fluorescence in the presence of saturating amounts of Ca²⁺ and effectively “zero Ca²⁺ respectively, K_{d} is the dissociation constant of Ca²⁺ from fura 2; and D is the ratio of fluorescence of fura 2 at 380 nm in zero Ca²⁺ and saturating amounts of Ca²⁺. Unless otherwise stated, measurements of peak [Ca²⁺] were made at the end of diastole. High-contrast microspheres attached to the cell surface of the cardiomyocytes were imaged using a charge-coupled device video camera attached to the microscope, and motion along a selected raster line segment who quantified by a video motion detector system (IONOPTIX). As shown in FIG. 4A, cardiomyocytes infected with Ad.RSV.βgal did not affect the Ca²⁺ transient or shortening compared with control uninfected cardiomyocytes. As depicted in FIG. 4B, the Ca²⁺ transient and shortening were significantly altered with increasing concentrations of Ad.RSV.PL (multiplicity of infection of 1, 10, and 100 pfu/cell): observed changes included prolongation of the Ca²⁺ transient and shortening and a decrease in the peak Ca²⁺. These results, summarized in Table 1, show that there was a dose-dependent prolongation of the Ca²⁺ transient and mechanical shortening up to 10 pfu/cell, with no further significant prolongation at 100 pfu/cell, with no further significant prolongation at 100 pfu/cell.

TABLE 1
Physiological Parameters of Cardiomyocytes
Overexpressing Phospholamban

| | | | Ad.RSV | |
|---|---|---|---|---|
| | | | PL | MOI = |
| | Uni- | MOI = 1 | MOI = 10 | 100 |
| | fected | pfu/Cell | pfu/Cell | pfu/Cell |
| Time to 80% relaxation | 344 ± 26 | 612 ± 38° | 710 ± 58 | 683 ± 50 |
| of the (Ca2+) m? | | | | |
| Time to 80% relaxation | 387 ± 22 | 544 ± 27° | 780 ± 44 | 798 ± 43 |
| of shortening, m? | | | | |
| Peak [Ca2+], μmol/L | 967 ± 43 | 798 ± 23° | 630 ± 33 | 590 ± 34 |
| n | 10 | 8 | 12 | 8 |

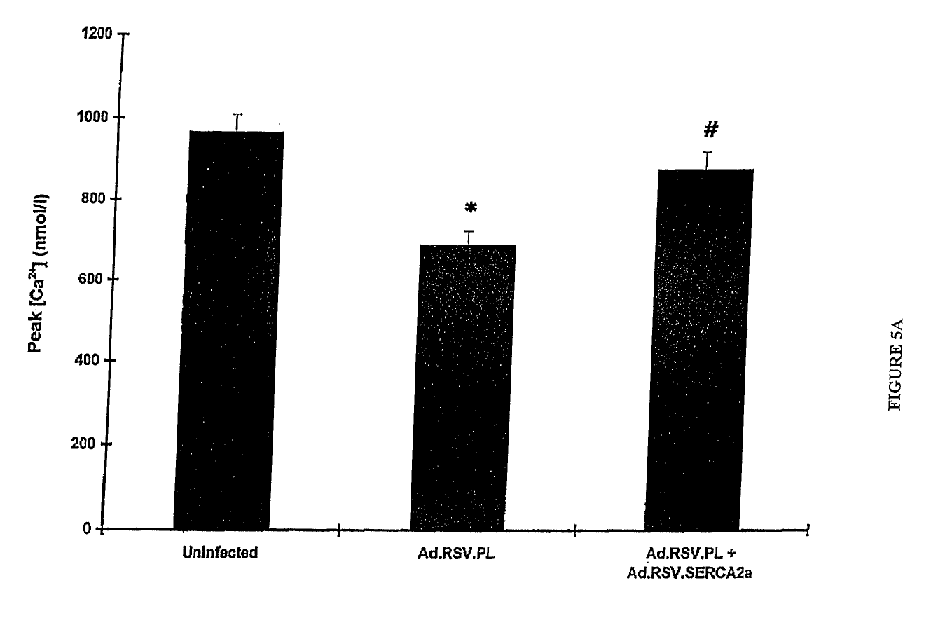

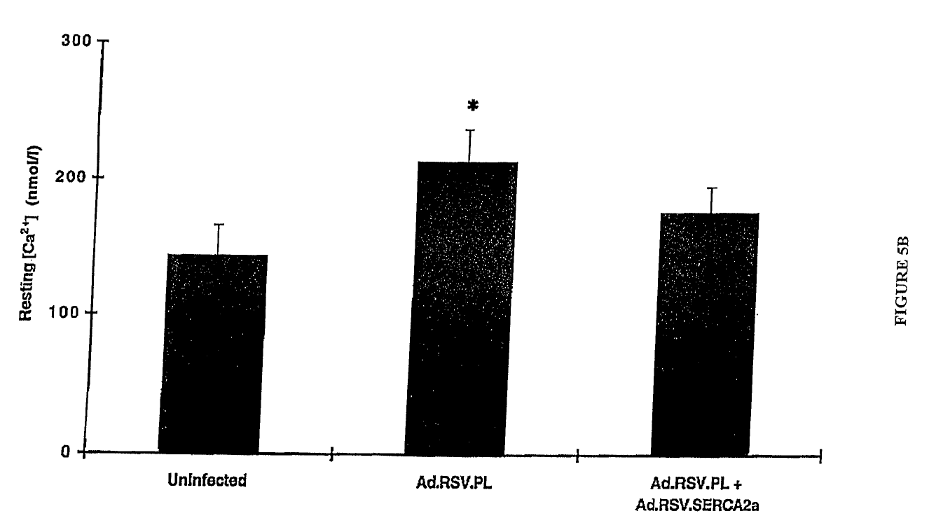

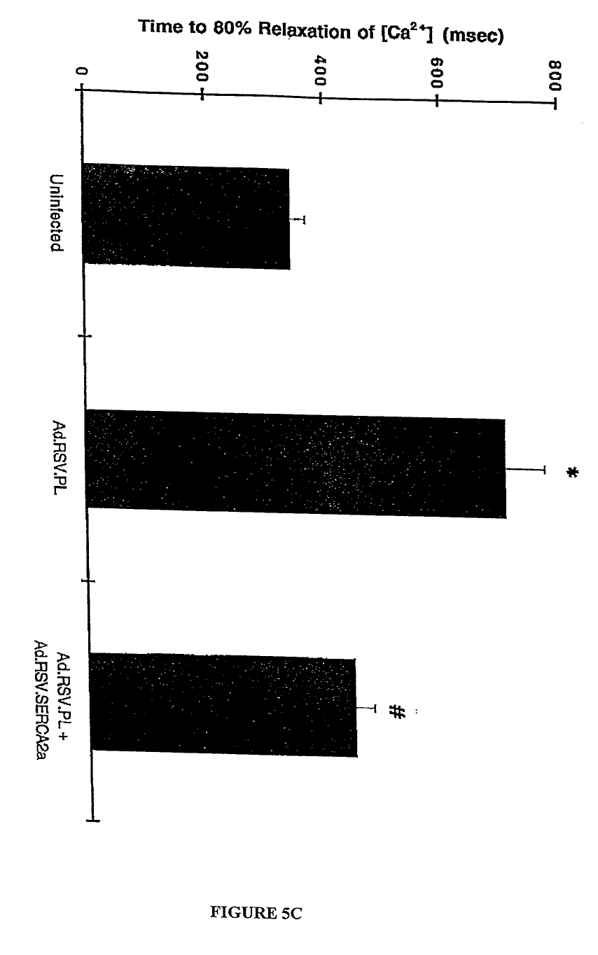

Similarly, peak [Ca²⁺] decreased up to 10 pfu/cell, with no further decrease at 100 pfu/cell. Coinfection with Ad.RSV.SERCA2a (multiplicity of infection of 10 pfu/cell) restored both the Ca²⁺ transient and the shortening to near normal levels, as shown in FIG. 4C. FIG. 5 shows a significant decrease in mean peak [Ca²⁺], a significant increase in mean resting [Ca²⁺], and a significant prolongation of the Ca²⁺ transient in the group of cardiomyocytes infected with Ad.RSV.PL (multiplicity of infection of 10 pfu/cell) compared with uninfected cells (panels a through c, respectively). These effects were partially restored by the addition of Ad.RSV.SERCA2a (multiplicity of infection of 10 pfu/cell) (FIG. 5). Similarly, the time course of shortening was significantly prolonged in cardiomyocytes infected with Ad.RSV.PL at a multiplicity of infection of 10 pfu/cell (time to 80% relaxation, from 387±22 to 780±44 milliseconds; P<0.5; n=12), whereas coinfection with Ad.RSV.SERCA2a restored the time course to normal (405±25 milliseconds, n=10, P>0.1 compared with uninfected cells).

Adenoviral gene transfer of phospholamban provides an attractive system for further elucidation of the effects of inhibiting SR Ca²⁺-ATPase on intracellular Ca²⁺ handling. A decrease in SRCa²⁺ uptake rates is expected to lead to a smaller amount of Ca²⁺ sequestered by the SR, resulting in a smaller amount of Ca²⁺ release. In neonatal cardiomyocytes, a significantly prolonged Ca²⁺ transient and a higher resting (Ca²⁺) was observed reflecting the decreased Ca²⁴ uptake and a decrease in peak (Ca²⁺) levels reflecting less Ca²⁺ available for release. These results show that the SR Ca²⁴-ATPase is important during relaxation by controlling the rate and amount of CA²⁺ sequestered and during contraction by releasing the Ca²⁺ that is taken up by the SR. Overexpression of both phospholamban and SERCA2a partially restored the Ca²⁺ transient; however, the time course of the Ca²⁺ transient was still prolonged in cardiomyocytes infected with both Ad.RSV.SERCA2a and Ad.RSV.PL. This finding was somewhat surprising, since the SR Ca²⁺-ATPase activity was restored to normal and even enhanced in cardiomyocytes infected with both Ad.RSV.SERCA2a and Ad.RSV.PL.

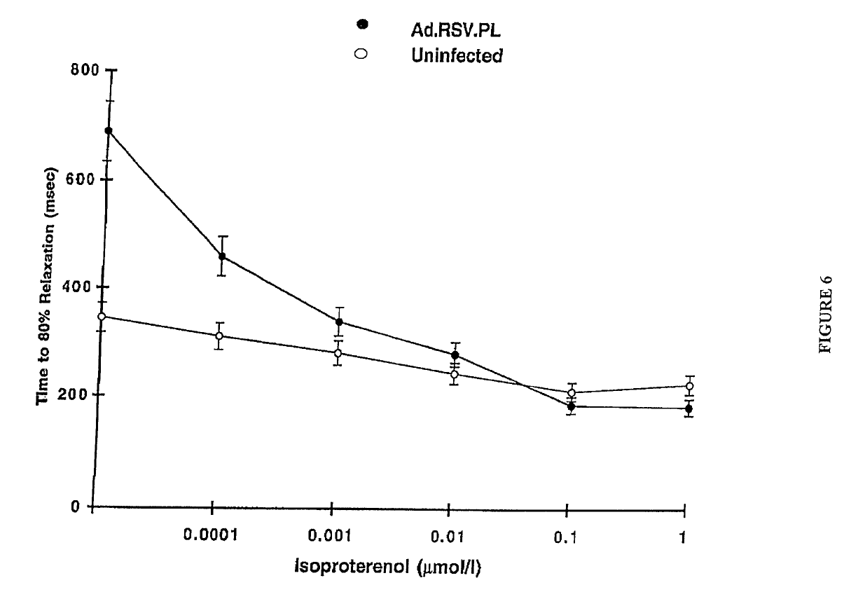

Phospholamban has been shown to play a key role in modulating the response of agents that increase cAMP levels in cardiomyocytes. Since phosphorylation of phospholamban reduces the inhibition to the SR Ca²⁺ pump, thereby enhancing the SR Ca²⁺-ATPase, we were specifically interested in evaluating the effects of β-agonism on the relaxation phase of the Ca²⁺ transient. In the basal state, the overexpression of phospholamban significantly prolongs the Ca²⁺ transient. As shown in FIG. 6, at maximal isoproterenol stimulation, the time course of the Ca²⁺ transients in the uninfected cardiomyocytes and the cardiomyocytes infected with Ad. RSV.PL were decreased to the same level. These findings show that phospholamban plays a major role in the enhanced relaxation of the heart to β-agonism. In addition, it corroborates these findings that phospholamban decreases the affinity of the SR Ca²⁺ pump for Ca²⁺ but does not decrease the maximal Ca²⁺ uptake rate.

4. Preparation of SR Membranes from Isolated Rat Cardiomyocytes

To isolate SR membrane from cultured cardiomyocyes, a procedure modified from Harigaya et al., Circ. Res., 1969, 25:781-794, as well as, Wienzek et al., 1992, 23:1149-1163, the contents of which are incorporated herein by reference, was used. Briefly, isolated neonatal cardiomyocytes were suspended in a buffer containing (mmol/L) sucrose 500, phenylmerhyisulfonyl fluoride 1 and PIPES 20, at pH 7.4. The cardiomyocytes were then disrupted with a homogenizer. The homogenates were centrifuged at 500 g for 20 minutes. The resultant supernatant was centrifuged at 25,000 g for 60 minutes to pellet the SR-enriched membrane. The pellet was re-suspended in a buffer containing (mmol/L) KCl 600, sucrose 30, and PIPES 20, frozen in liquid nitrogen, and stored at −70° C. Protein concentration was determined in these preparations by a modified Bradford procedure, described in Bradford et al., Anal. Biochem., 1976, 72:248-260, the contents of which are incorporated herein by reference, using bovine scrum albumin for the standard curve (Bio-Rad).

5. Western Blot Analysis of Phospholamban and SERCA2a in SR Preparations

SDS-PAGE was performed on the isolated membranes from cell cultures under reducing conditions on a 7.5% separation gel with a 4% stacking gel in a Miniprotean II cell (Bio-Rad). Proteins were then transferred to a Hybond-ECL nitrocellulose for 2 hours. The blots were blocked in 5% nonfat milk in Tris-buffered saline for 3 hours at room temperature. For immunoreaction, the blot was incubated with 1:2500 diluted monoclonal anti-SERCA2 antibody (Affinity BioReagents) or 1:2500 diluted anti-cardiac phospholamban monoclonal IgG (UBI) for 90 minutes at room temperature. After washing, the blots were incubated in a solution containing peroxidase-labeled goat anti-mouse IgG (dilution, 1:1000) for 90 minutes at room temperature. The blot was then incubated in a chemiluminescence system and exposed to an X-OMAT x-ray film (Fuji Films) for 1 minute. The densities of the bands were evaluated using NIH Image. Normalization was performed by dividing densitometric units of each membrane preparation by the protein amounts in each of these preparations. Serial dilution of the membrane preparations revealed a linear relationship between amounts of protein and the densities of the SERCA2a immunoreactive hands (data not shown).

6. SR Ca²⁺-ATPase Activity

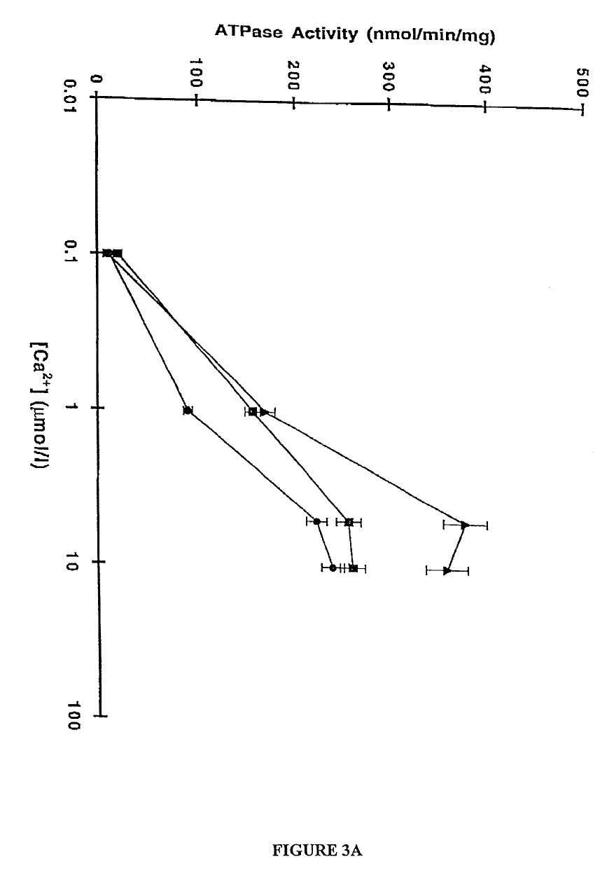

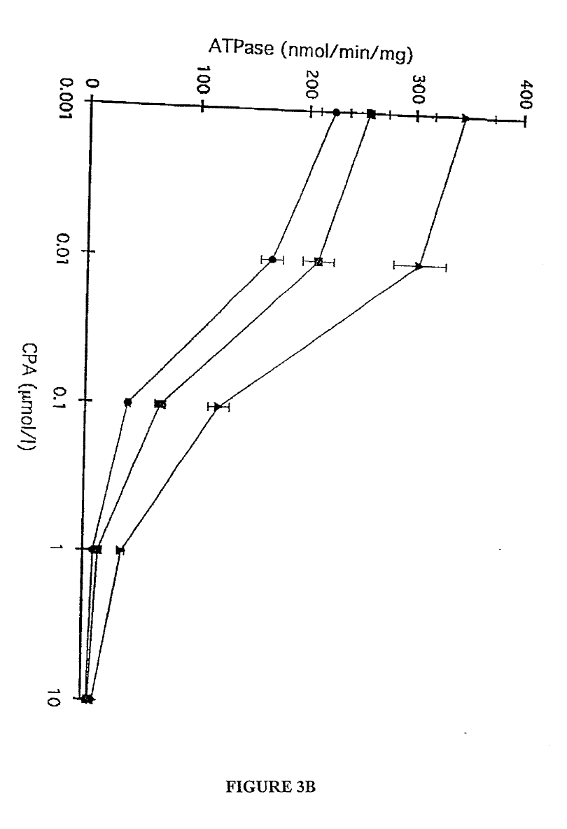

SR Ca²⁺-ATPase activity assays were carried out according to Chu A. et al., Methods Enzymol., 1988, 157:36-46, the contents of which are incorporated herein by reference, on the basis of pyruvate/NADH-coupled reactions. By use of a photomotor (Beckman DU 640) adjusted at a wavelength of 540 nm, oxidation of NADH (which is coupled to the SR Ca²⁺-ATPase) was assessed at 37° C. in the membrane preparations by the difference of the total absorbance and basal absorbance. The reaction was carried out in a volume of 1 mL. All experiments were carried out in triplicate. The activity of the Ca²⁺-ATPase was calculated as follows: Δabsorbance/6.22×protein×time (in nmol ATP/mg protein×min). The measurements were repeated at different [Ca²⁺] levels. The effect of the specific Ca²⁺-ATPase inhibitor CPA at a concentration range of 0.001 to 10 μmol/L was also studied in those preparations, as described in Schwinger et al., Circulation, 1995, 92:3220-3228 and Baudet et al., Circ. Res., 1993, 73:813-819, the contents of which are incorporated herein by reference. As shown in FIG. 3A, the relationship between ATPase activity and Ca²⁺ was shifted to the right in the preparations from cardiomyocytes overexpressing phospholamban compared with the uninfected preparations without changing maximal Ca²⁺-ATPase activity. Coinfection with Ad.RSV.SERCA2a restored the CA²⁺-ATPase activity and also increased the maximal Ca²⁺-ATPase activity. To verify that the ATPase activity measured from the membrane preparations was SR-related, the specific inhibitor CPA was used after maximally activating the SR Ca²⁺-ATPase with 10 μmol/L of Ca²⁺. As shown in FIG. 3B, CPA inhibited the SR Ca²⁺-ATPase activity in a dose-dependent fashion in all three membrane preparations (uninfected, Ad.RSV.PL, and d.RSV.PL+Ad.RSV.SERCA2a).

The SR Ca²⁺-ATPase plays a key role in excitation-contraction coupling, lowering Ca²⁺ during relaxation in cardiomyocytes, and “loading” the SR with Ca²⁺ for the subsequent release and contractile activation. The Ca²⁺-pumping activity of this enzyme is influenced by phospholamban. In the unphosphorylated state, phospholamban inhibits the Ca²⁺-ATPase, whereas phosphorylation of phospholamban by cAMP-dependent protein kinase and by Ca²⁺ calmodulin-dependent protein kinase reverses this inhibition. Therefore, an increase in phospholamban content should decrease the affinity of the SR Ca²⁺ pump for Ca²⁺. As shown in FIG. 4, overexpression of phospholamban shifted the relationship between SR Ca²⁺-ATPase activity and Ca²⁺ to the right, indicating a decrease of the sensitivity of the SR Ca²⁺ to pump to Ca²⁺. However, there was no change in the maximal Ca²⁺-ATPase activity in the Ad.RSV.PL-infected cardiomyocytes. This shows that the Vₘₐₓ of the Ca²⁺-ATPase of cardiac SR is not altered by interaction with phospholamban and phosphorylation, and that in mice overexpressing phospholamban, the affinity of the SR Ca²⁺ pump for Ca²⁺ was decreased but that the maximal velocity of the SR Ca²⁺ uptake was not changed. From the present experiment, it can also be concluded that phospholamban affects the affinity of the SR Ca²⁺ pump for CA²⁺ without changing the maximal ATPase activity. The concomitant overexpression of SERCA2a and phospholamban restored the ATPase activity and also increased the maximal Ca²⁺-ATPase activity. This brings further evidence that the expression of additional SR Ca²⁺-ATPase pumps can overcome the inhibitory effects of phospholamban.

7. Statistical Analyses

Data were represented as mean±SEM for continuous variables. Stutent's test was used to compare the means of normally distributed continuous variables. Parametric one-way ANOVA techniques were used to compare normally distributed contiguous variables among uninfected groups of cells, Ad.RSV.βgal-infected cells, Ad.RSV.PL-infected cells, and Ad.RSV.SERCA2a-infected cells.

8. Adenoviral Somatic Gene Transfer

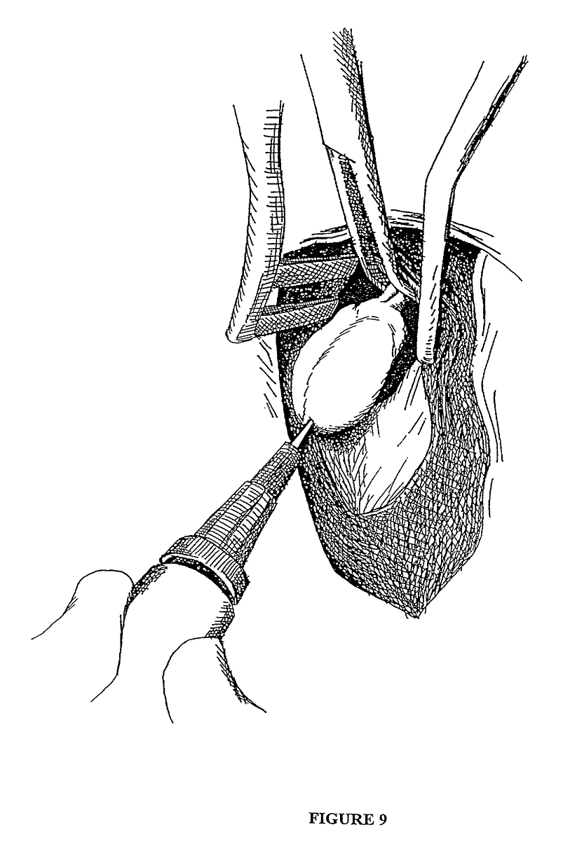

Rats and mice were anesthetized with intraperitoneal pentobarbital and placed on a ventilator. The chest was entered form the left side through the third intercostal space. The pericardium was opened and a 7-0 suture placed at the apex of the left ventricle. The aorta and pulmonary artery were identified. A 22 G catheter containing 200 μl of adenovirus was advanced from the apex of the left ventricle to the aortic root. The aorta and pulmonary artery were clamped distal to the site of the catheter and the adenovirus solution was injected as shown in FIG. 9. The clamp was maintained for 10 seconds while the heart was pumping against a closed system (isovolumically). This allowed the adenovirus solution to circulate down the coronary arteries and perfuse the whole heart without direct manipulation of the coronaries. After the 10 seconds, the clamp on the aorta and the pulmonary artery was released, the chest was evacuated from air and blood and closed. Finally, the animals were taken off the ventilator.

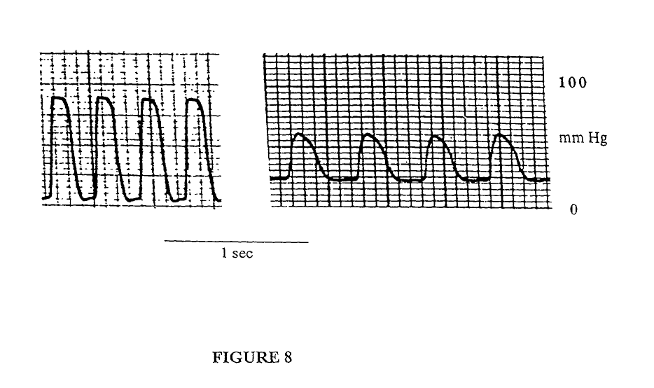

The expression pattern seen after direct injection is localized whereas the catheter-based technique is essentially homogeneous. The pressure tracing of the Ad.PL transduced hearts displayed a markedly prolonged relaxation and reduced pressure development as shown in FIG. 8.

9. Gene Transfer of the Sarcoplasmic Reticulum Calcium ATPase Improves Left Ventricular Function in Aortic-banded Rats in Transition to Failure

In human and experimental models of heart failure, sarcoplasmic reticulum Ca²⁺ ATPase (SERCA2a) activity has been shown to be significantly decreased. In this example the ability of SERCA2a expression to improve ventricular function in heart failure was investigated by creating an ascending aortic constriction in 10 rats. After 20-24 weeks, during the transition from left ventricular hypertrophy to failure, 200 μl of a solution containing 5×10⁹ plaque forming units of replication-deficient adenovirus carrying SERCA2a (Ad.SERCA) (n=4) or the reporter gene β-galctosidase (Ad.βgal) (n=6) were injected intracoronary via the catheter-based technique described supra. Two days after the procedure, the rats underwent open chest measurement of left ventricular pressure. Heart rate (HR), left ventricular end-diastolic pressure (LVEDP), and left ventricular systolic pressure (LVSP) were measured. Peak +dP/dt and −dP/dt were calculated. As shown in Table 2, the magnitudes of peak +dP/dt and −dP/dt which are indices of systolic and diastolic function were markedly increased in hearts transduced with the SERCA2a carrying adenovirus. Therefore, this example indicates that overexpression of SERCA2a in a rat model of pressure-overload hypertrophy in transition to failure improved left ventricular systolic and diastolic function.

TABLE 2

| | | | | +dP/dt | −dP/dt |
|---|---|---|---|---|---|
| | HR | LVEDP | LVSP | (mmHg/ | (mmHg/ |
| | (bpm) | (mmHg) | (mmHg) | sec) | sec) |
| Ad.βgal | 416 ± | 6 ± 4 | 114 ± 16 | 5687 ± | −5023 ± |
| | 46 | | | 1019 | 1803 |
| Ad.SERCA | 450 ± | 9 ± 3 | 148 ± 40 | 9631 ± | −8385 ± |
| | 53 | | | 3568# | 980# |

#p < 0.05 compared to Ad.βgal

10. Gene Transfer of Antisense of Phospholamban Improves Contractility in Isolated Cardiomyocytes in Rat and Human

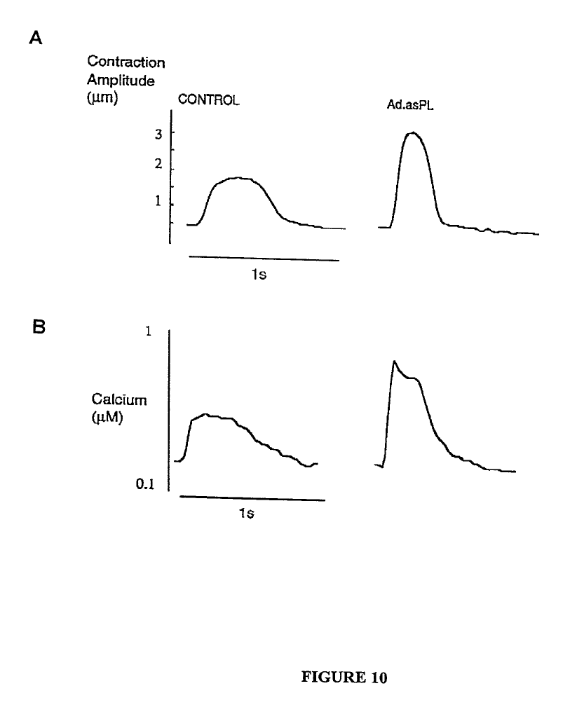

A. Delayed cardiac relaxation in failing hearts is attributed to a reduced activity of the Sarcoplasmic Reticulum Calcium ATPase. Phospholamban inhibits SERCA2a activity and is, therefore, a potential target to improve cardiac function. In this Example, an adenovirus carrying the full length antisense cDNA of phospholamban (Ad.asPL) was constructed using the methods described above. This construct was then used to infect neonatal cardiac myocytes as described in Example 3. As indicated in FIG. 10A, infection of neonatal cardiac myocytes with the Ad.asPL construct increased the contraction amplitude and significantly shortened the time course of the contraction. The adenovirus-mediated gene transfer of the antisense cDNA for phospholamban also resulted in a modification of intracellular calcium handling and shortening in myocardial cells (see FIG. 10B) indicating that such vectors can be used for increasing the contractility of myocardial cells in heart failure.

B. Since human heart failure are mainly due to coronary artery disease or idiopathic in nature, we ablated phosholamban by antisense strategies using adenoviral gene transfer in isolated ventricular cardiac myocytes from eight patients with end-stage heart failure of various etiologies (idiopathic, ischemic and hypertrophic). The co-expression of green fluorescent protein GFP allowed us to identify the cells that were infected and expressing the transgene after 48 hours.

Following isolation, failing human cardiomyocytes were infected with an adenovirus carrying antisense phospholamban. Forty-eight hours after infection a cardiomyocyte is visualized with white light and at 510 nm with single excitation peak at 490 nm of blue light. Co-expression of GFP demonstrated visually the ablation of phospholamban in the cell. Recordings were performed from cardiomyocytes isolated from a donor nonfailing heart and from a failing heart infected with either an adenovirus expressing green fluorescent protein, Ad.GFP or carrying the antisense of phospholamban, Ad.asPL, stimulated at 1 Hz at 37° C. The failing cell had a characteristic decrease in contraction and prolonged relaxation along with a prolonged Ca*′transient. Ablation of phospholamban in the failing cardiomyocyte normalized these parameters. Ablation of phospholamban in failing cardiomyocytes induced a faster contraction velocity (15.4±2.7 vs 6.9±2% shortening/sec, p=0.008)and enhanced relaxation velocity (18.6±4.4 vs 6.6±3.7, p=0.01).

These results show that regardless of etiology, in human heart failure, improving calcium cycling by decreasing phospholamban inhibition to SERCA2a, restores contractility in failing ventricular cells of different etiologies'. These findings also extend previous results that overexpression of SERCA2a improves contractile function in human failing cardiac myocytes. Finally these findings underscore the importance of validating experimental results from murine models in relevant human tissues.

11. Gene Transfer of the Sarcoplasmic Reticulum Calcium ATPase Improves Survival in Aortic-banded Rats in Transition to Failure

Pharmacological agents that increase contractility have been repeatedly shown to worsen survival in patients with congestive heart failure and to increase the energetic requirements on the heart (O'Connor et el. (1999). Am Heart J 138(1 Pt 1):78-86). Since the heart performs uninterrupted biochemical and mechanical work, it requires a continuous supply of energy in the form of ATP by mostly oxidative metabolism under normal conditions with major energy reserve molecule represented by phosphocreatine (PCr). In the normal heart, although the majority (60%) of the energy consumption is due to cross-bridge cycling, relaxation requires an energy expenditure of 15% to remove Ca⁺⁺ from the cytoplasm. This high level of free energy |ΔGp| required by the SERCA2a reaction is directly related to the magnitude of the Ca²⁺ gradient across the SR (Tian et al. (1998) Am J Physiol 275(6 Pt 2):H2064-71). Failing hearts have a reduced ratio PCr/ATP in human as well as in animal models of heart failure so that less energy reserve is available for the cellular processes. This decrease in energy reserve has been shown to be by itself a predictor of mortality in patients with dilated cardiomyopathy (Neubauer et al. (1997) Circulation 96(7):2190-6).

In this Example, unlike other pharmacologic agents that increase inotropy, reconstitution of normal levels of SERCA2a by adenoviral gene transfer improves contractile performance as well as survival in aortic banded rats with developed heart failure without adversely affecting energetics possibly by reducing the intracellular diastolic Ca²⁺ overload.

Experimental Protocols

A. Construction of Recombinant Adenoviruses

We constructed an adenovirus containing SERCA2a and GFP controlled by separate CMV promoters (Ad.SERCA2a). An adenovirus containing both β-galactosidase and GFP controlled by separate CMV promoters (Ad.βgal-GFP) was used as control as described earlier (Haq et al. (2000) J Cell Biol 151(1):117-130). The titer of stocks used for these studies measured by plaque assays were: 3×10¹¹ pfu/ml for Ad.βgal-GFP and 1.8×10¹¹ pfu/ml for Ad.SERCA2a with a particle/pfu ratio of 8:1 and 18:1 respectively (viral particles/ml determined using the relationship one absorbance unit at 260 nm is equal to 10¹² viral particles/ml). These recombinant adenoviruses were tested for the absence of wild-type virus by PCR of the early transcriptional unit E1.

B. Aortic Banding

Four-week old Sprague Dawley rats (70-80 g) were obtained from Taconic Farms. After 2-3 days of acclimatization, the rats were anesthetized with intraperitoneal pentobarbital (65 mg/kg) and placed on a ventilator. A suprasternal incision was made exposing the aortic root and a tantalum clip with an internal diameter of 0.58 mm (Weck, Inc.) was placed on the ascending aorta. Animals in the sham group underwent a similar procedure without insertion of a clip. The supraclavicular incision was then closed and the rats were transferred back to their cages. The supraclavicular approach was performed because during gene delivery a thoracotomy is necessary and by not opening the thorax during the initial aortic banding avoids adhesions when gene delivery is performed thereby decreasing the morbidity of the procedure.

Animals were initially divided into two groups: one group of 45 animals with aortic banding and a second group of 42 animals which were sham-operated. Three animals did not survive the initial operation in the aortic banding group and 2 animals did not survive in the sham-operated group. In the animals which were aortic banded we waited 26-28 weeks for the animals to develop left ventricular dilatation prior to cardiac gene transfer. In this last group as well as in the sham-operated group, fourteen animals did not undergo gene transfer and were followed longitudinally. The rest of the animals underwent adenoviral gene transfer with either Ad.SERCA2a or Ad.bgal-GFP.

C. ³¹P NMR Measurements

NMR Spectroscopy

Stable energetic state in rat hearts was confirmed from ³¹P NMR signals of phosphocreatine, ATP, and inorganic phosphate as described in Lewandowski et al. ((1995) American J Physiol 269(1 Pt 2):H160-8). NMR data was collected on a Bruker 400 MHz spectrometer interfaced to a 9.4 tesla, vertical bore, superconducting magnet. ³¹ P spectra were obtained from isolated hearts perfused within a broad-band, 20 mm NMR probe (Bruker Instruments). ³¹P-NMR spectra were acquired in 128 scans using a 161 MHz, 450 excitation pulse, a 1.8s repetition time, 35 ppm sweep width, and 8 K data set. Post processing of the summed free induction decay (FID's) NMR data included 20 Hz line broadening, Fourier transformation, and phase correction. Peak assignments were referenced to the well established resonance signal of PCr at 0 ppm, with identification and assignment of the α, β, and γ phosphate signals of ATP. Signal intensity was determined using NMR-dedicated data analysis.

Isolated, perfused rat heart preparation:

Hearts were retrograde perfused from a 100 cm hydrostatic perfusion column with modified Krebs-Henseleit buffer (116 mM NaCl, 4 mM KCl, 1.5 mM CaCl₂, 1.2 mM MgSO₄, 1.2 mM NaH₂PO₄, and 25 mM NaHCO₃, equilibrated with 95% O₂/5% CO₂ at 37° C.) that contained 5 mM glucose in a 2 liter reservoir. A polyethylene catheter was inserted into the pulmonary artery allowing collection of coronary effluent for measurement of oxygen consumption with a blood-gas analysis machine. Hearts spontaneously beat, contracting against a fluid-filled intraventricular balloon connected to a pressure transducer and inflated to an end diastolic pressure of 5 mm Hg. The isolated hearts were placed in a borosilicate glass vial. A 10-15 ml volume of coronary effluent bathed the heart. Temperature was maintained at 37° C. with both perfusate temperature and a thermal control unit interfaced to the NMR system.

D. Serial Echocardiographic Assessment

After eighteen weeks of banding, serial echocardiograms were performed on a weekly basis. Animals were anesthetized with pentobarbital 40 mg/kg intra-peritoneally, and the anterior chest shaved. Transthoracic M-mode and two-dimensional echocardiography was performed with a Hewlett-Packard Sonos 5500 imaging system (Andover, Mass.) with a 12 MHz broadband transducer. A mid-papillary level left ventricular short axis view was used and the images were stored digitally. Measurements of posterior wall thickness, left ventricular diastolic dimension and fractional shortening were performed off-line. The epicardial surface of the anterior wall was not reliably visualized in all animals. Gene transfer was performed in all animals within 3 days of detection of a drop in fractional shortening of >25% compared to the fractional shortening at 18 weeks post-banding. In the sham operated rats, gene delivery was performed at 27 weeks.

E. Adenoviral Delivery Protocol

The group of animals subjected to aortic banding were further subdivided in three additional groups of sixteen, twelve, and fourteen receiving respectively Ad.SERCA2a, Ad.bgal-GFP, or no adenovirus. The group of sham-operated animals was also subdivided into three groups of fourteen, twelve, and fourteen Ad.SERCA2a, Ad.bgal-GFP, or no adenovirus. The adenoviral delivery system has been described in Miyamoto et al. ((2000) Proc Natl Acad Sci USA 97(2):793-8). Briefly, after anesthetizing the rats and performing a thoracotomy, a 22 G catheter containing 200 ml of adenoviral solution (10¹⁰ pfu) was advanced from the apex of the left ventricle to the aortic root. The aorta and main pulmonary artery were clamped for 20 seconds distal to the site of the catheter and the solution injected, then the chest was closed, the animals were extubated and transferred back to their cages.

F. Measurements of Left Ventricular Volume & Elastance

Prior to euthanasia, rats in the different treatment groups were anesthetized with 65 mg/kg of pentobarbital and mechanically ventilated. After thoracotomy, a small incision was then made in the apex of the left ventricle and a 1.4 French high fidelity pressure transducer (Millar Instruments, TX) introduced into the left ventricle. Pressure measurements were digitized at 1.0 kHz and stored for further analysis using commercially available software (Sonolab, Sonometrics Co., Alberta, Canada) and four 0.7 mm piezoelectric crystals (Sonometrics Co., Canada) were placed over the surface of the left ventricle along the short axis of the ventricle at the level of the mitral valve and at the apex of the left ventricle to measure the inter-crystal distances. The left ventricular volume was derived using a mathematical model using CARDIOSOFT (Sonometrics Co., Canada). Left ventricular pressure-volume loops were generated under different loading conditions by clamping the inferior vena cava. The end-systolic pressure-volume relationship was obtained by producing a series of pressure dimension loops over a range of loading conditions and connecting the upper left hand comers of the individual pressure-dimension loops to generate the maximal slope.

G. Western Blot Analysis

SDS-PAGE was performed on the tissue lysate under reducing conditions on 7.5% separation gels with a 4% stacking gel in a Miniprotean II cell (BIORAD). Proteins were then transferred to a Hybond-ECL nitrocellulose for 2 hours and blocked in 5% nonfat milk for 3 hours. For immunoreaction, the blots were incubated with 1:2,500 diluted monoclonal antibodies to either SERCA2a (MA3-919; Affinity BioReagents, CO), or 1:1,000 diluted anti-calsequestrin (MA3-913; Affinity Bioreagents) for 90 minutes at room temperature. After washing the blots were exposed for 1 hour to HRP conjugated anti mouse antibody for chemo-luminescent detection.

H. SR Ca²⁺ ATPase Activity

SR Ca²⁺ ATPase activity assays were carried out based on a Pyruvate/NADH coupled reactions as previously described (Miyamoto, supra). Using a photometer (Beckman DU 640) adjusted at a wavelength of 340 nm, oxidation of NADH (which is coupled to the SR Ca²⁺-ATPase) was assessed at 37° C. in triplicates at different [Ca²⁺]. The reaction was carried out in a volume of 1 ml. Ca²⁺-ATPase activity was calculated as: Δ Absorbence/(6.22×protein×time) in nmol ATP/(mg protein×min).

I. Statistics

All values are presented as mean±sd. A two-factor ANOVA was performed to compare the different hemodynamic parameters among the different groups. For the echocardiography data, where the variables were examined at various intervals, ANOVA with repeated measures was performed. Comparison of survival in the different groups of animals was analyzed by a log-rank test with the Kaplan-Meier method. Statistical significance was accepted at the level of p<0.05.

Effect on Survival

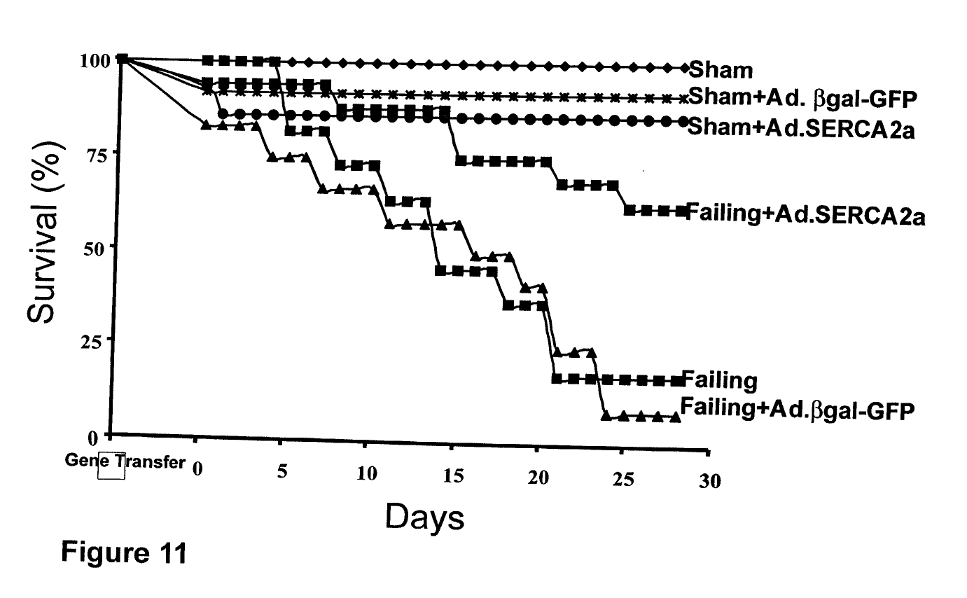

FIG. 11 shows the survival curve for the six different groups studied. The sham operated animals did not show any premature mortality. The sham operated animals that were either infected with Ad.bgal-GFP or Ad.SERCA2a had early mortalities related to the surgical intervention of cardiac gene transfer, but then the survival curves leveled off for both sham+ Ad.bgal-GFP and sham+Ad.SERCA2a. In the failing group, the non-infected animals had a survival curve that decreased steadily and at 4 weeks the survival rate was only 18% (p<0.0005 compared to sham). In the failing group+ Ad.bgal-GFP the survival curve also decreased and at 4 weeks the survival rate was only 9% (p<0.001 compared to sham+ Ad.bgal-GFP). However in the failing group+Ad.SERCA2a, the survival curve was significantly improved compared to failing+Ad.SERCA2a (p<0.001 compared to failing+ Ad.bgal-GFP).

Characterization of Animals

Following 18 weeks of aortic banding, the animals showed echocardiographic signs of left ventricular hypertrophy including an increase in wall thickness (both posterior and septal), an increase in posterior wall thickness, a decrease in left ventricular dimensions and an increase in fractional shortening as shown in Table 3. Of note at that time the animals showed no clinical signs of heart failure. After 26-27 weeks of banding, these animals had uniformly 1) small pericardial effusions, 2) pleural effusions, 3) an increase in lung weight, 4) ascites, and 5) dyspnea at rest all indicative signs of developed heart failure. Echocardiographically, LV end-diastolic dimensions increased and fractional shortening decreased.

TABLE 3
Echocardiographic Measures in Rats after Sham Surgery or Aortic
Banding

| | Septum | PW | LVEDD | LVESD | FS |
|---|---|---|---|---|---|
| | (mm) | (mm) | (mm) | (mm) | (%) |
| Sham | 14.9 ± 1.1 | 13.5 ± 1.0 | 66.8 ± 3.8 | 40.4 ± 6.0 | 40.0 ± 6.3 |
| Aortic | 20.1 ± | 19.8 ± | 61.9 ± | 34.0 ± | 46.0 ± |
| banding | 3.9ˆ | 2.8ˆ | 6.4*ˆ | 6.2*ˆ | 8.2*ˆ # |
| (18 weeks) | | | | | |
| Aortic | 19.7 ± | 18.5 ± | 69.5 ± | 45.1 ± | 36.0 ± |
| banding | 2.8‡ | 2.3‡ | 6.3# | 6.9ˆ | 10.4# |
| (27 weeks) | | | | | |

PW: posterior wall thickness during diastole LVDD: Left ventricular Diameter during diastole LVSD: Left ventricular Systolic Diameter during Systole FS: Fractional shortening* p < 0.0005 vs Aortic banding (27 weeks)‡ p < 0.005, ˆ p < 0.005, #p < 0.05 vs control

Cardiac Gene Transfer & SERCA2a Expression

We first examined the expression of SERCA2a 28 days following adenoviral gene transfer. There was a decrease in SERCA2a in failing rats compared to sham operated rats. The protein expression of SERCA2a was decreased in failing rat left ventricles when compared to SERCA2a levels of sham left ventricles. Adenoviral gene transfer of SERCA2a in failing hearts increased SERCA2a protein expression restoring it to levels observed in the nonfailing hearts. The protein levels were normalized to calsequestrin which did not change among the different groups. To evaluate whether other tissues are infected we histologically examined sections of aorta, liver, and lung following infection with the cardiac specific Ad.SERCA2a. There was no evidence of SERCA2a expression in the aorta, in the liver and lungs. In the infected rat hearts there was no evidence of disruption of normal myocardial architecture or collagen deposition.

Thus, we restored SERCA2a protein to normal levels in failing hearts. In addition, we showed that the expression of SERCA2a to normal levels was sustained for up to four weeks. This seemed somewhat surprising since first generation adenoviruses induce transient expression peaking at 7-10 days and disappearing after 10 days 23. However, endogenous turnover of SERCA2a is about 14-15 days in young rats and longer in older rats 24 which would explain the sustained levels of SERCA2a.

SR Ca²⁺ ATPase Activity

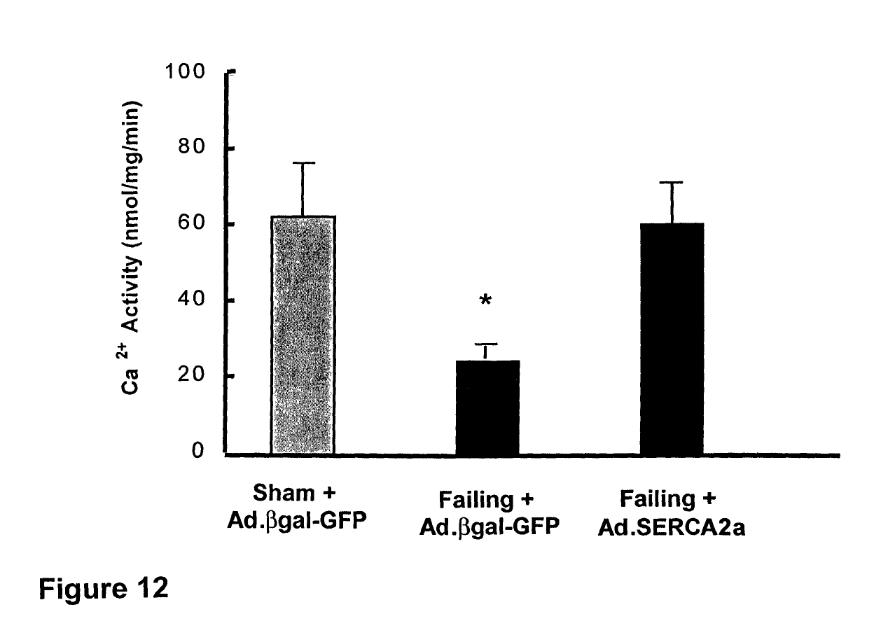

We measured SR ATPase activity at a calcium concentration of 10 mM in 1) sham+ Ad.bgal-GFP 2) failing+ Ad.bgal-GFP, and 3) failing+Ad.SERCA2a. As shown in FIG. 12, there was a decrease in maximal ATPase activity in the failing group. Gene transfer of SERCA2a restored ATPase activity back to normal levels in the failing group four weeks following gene transfer.

SERCA2a Expression and Cardiac Energetics

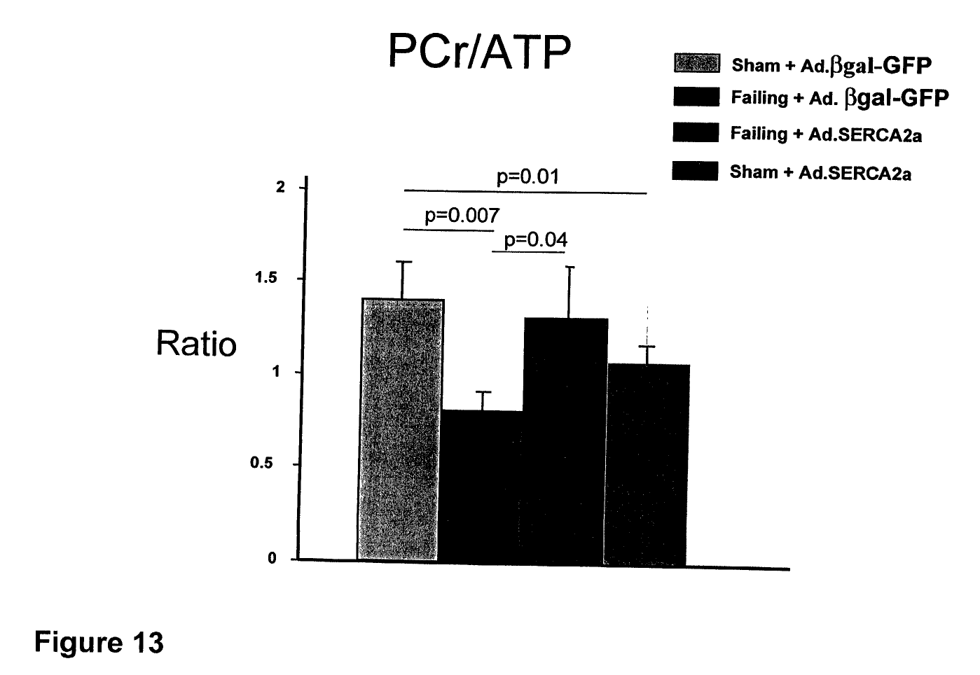

Representative ³¹P-NMR spectra obtained from three groups of rats: 1) sham+ Ad.bgal-GFP, 2) failing+ Ad.bgal-GFP, 3) failing+Ad.SERCA2a are shown in FIG. 13. These spectra show that the ratios of total amounts PCr to ATP are lower in the failing heart when compared with the sham heart. The integrated area for Pi was also increased in the failing heart. The overexpression of SERCA2a in failing heart restored and normalized both the content of PCr and ATP while the integrated area for Pi was reduced. Interestingly we found that overexpression of SERCA2a in sham operated animals induces a reduction in PCr:ATP ratio (FIG. 13).

Thus, restoring SERCA2a levels to normal induced an improvement in the creatine phosphate to ATP ratio. The findings of improved cardiac energetics in developed heart failure was somewhat surprising since overexpression of SERCA2a would be anticipated to increase ATP hydrolysis thereby driving creatine phosphate down. Indeed, this increase in ATP hydrolysis is consistent with our observation of reduced PCr/ATP in the group of sham-operated hearts that were overexpressing SERCA2a. These results are also consistent with previous results showing that PCr/ATP was decreased in the phospholamban-deficient hearts relative to the wild-type hearts (Chu et al. (1996) Circ Res 79(6):1064-76). In heart failure however, elevated calcium levels would increase energy demand. Furthermore, the thermodynamic reserve for the SR Ca²⁺-ATPase reaction is limited and in order to maintain the normal Ca²⁺ gradient, the SR Ca²⁺-ATPase reaction requires a |ΔGp| of at least 52 kJ/mol, 85-90% of it from ATP. Therefore, of all the ATPase reactions in cardiac myocytes, the SR Ca²⁺-ATPase reaction is the most vulnerable to a decrease in |ΔGp|.

Effects of SERCA2a Overexpression on LV Volumes and Elastance

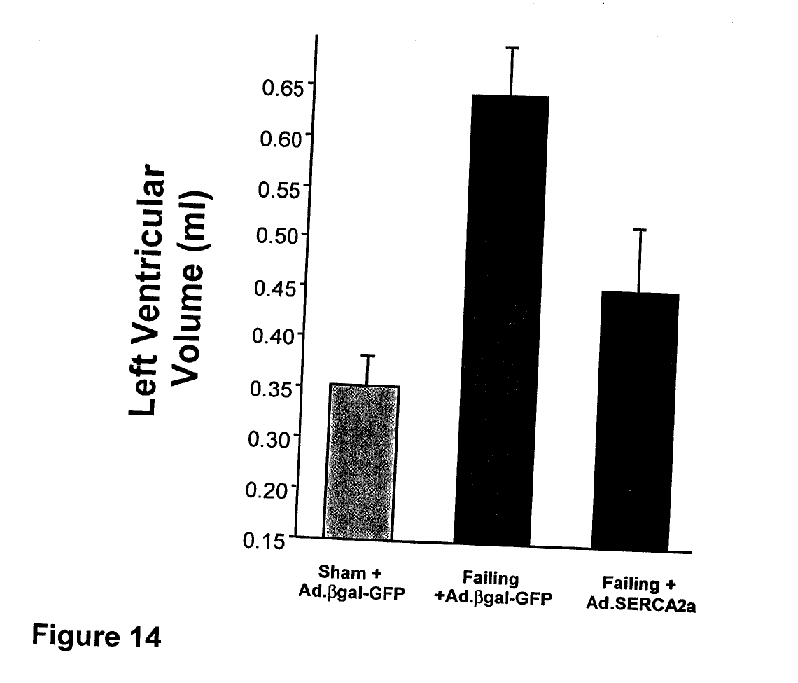

To determine left ventricular function, pressure-ventricular analysis was performed in a subset of animals. LV volumes were significantly increased in the failing rats (0.64±0.05 vs. 0.35±0.03 ml, p<0.02). Overexpression of SERCA2a normalized LV dimensions (0.46±0.07 ml) in the failing hearts (FIG. 14). To alter loading conditions, we clamped the inferior vena cava in the open-chested animals thereby reducing ventricular volume. This enabled us to calculate the end-systolic pressure volume relationship using a series of measurements made under varying pre-load conditions. The slope of the end-systolic pressure dimension relationship was lower in failing hearts infected with Ad.bgal-GFP compared to control indicating a diminished state of intrinsic myocardial contractility: 450±71 mmHg/ml vs 718±83 mmHg/mm (p<0.02). Overexpression of SERCA2a restored the slope of the end-systolic pressure dimension relationship to control levels (691±91 mmHg/ml, p<0.03 compared to failing+ Ad.bgal-GFP; p>0.1 compared to sham+ Ad.bgal-GFP).

Effect on Morphological Parameters

As shown in table 4, the failing hearts had a significant increase in heart mass when normalized to either tibial length or to body mass. Tibial length which was used as an index of growth independent of body weight was uniformly constant across the different groups. Body mass was also not significantly different across the different groups. Overexpression of SERCA2a in the failing heart did not have a significant effect on left ventricular mass whether normalized to tibial length or body mass.

TABLE 4
Morphometric Analyses

| | Sham + | Sham + | Failing + | Failing + |
|---|---|---|---|---|
| | Ad.βgal-GFP | Ad.SERCA2a | Ad.βgal-GFP | Ad.SERCA2a |
| HW/ | 3.7 ± | 4.4 ± | 4.4* ± | 43* ± |
| BW | 0.3 | 0.6 | 0.5 | 0.4 |
| ×10⁴ | | | | |
| HW/ | 44.8 ± | 55.3 ± | 50.8* ± | 50.3* ± |
| TL | 4.3 | 6.2 | 4.4 | 6.3 |
| ×10² | | | | |
| (g/ | | | | |
| mm) | | | | |

HW: heart weight BW: Body weight TL Tibial length* p < 0.05 compared to Sham + Ad.GFP

HW: heart weightBW: Body weightTL Tibial length*p<0.05 compared to Sham+Ad.GFP

Survival Following Gene Transfer

Herein, we show that restoration of SERCA2a expression by cardiac gene transfer in vivo improves not only contractile function but also survival and cardiac energetics. In addition, cardiac gene transfer of SERCA2a induced a reversal of adverse remodeling in the failing hearts.

In this model of heart failure SERCA2a overexpression improved parameters of inotropy and normalized contractile reserve. These effects translate into an inotropic intervention. However, other inotropic interventions have been shown clinically to increase mortality in chronic heart failure in numerous trials (Stevenson (1998) New England Journal of Medicine 339(25):1848-50). There are however significant differences between increasing inotropy with pharmacological agents that usually increase cAMP and enhancing inotropy with the overexpression of SERCA2a. Unlike agents that increase cAMP, thereby increasing intracellular Ca²⁺, reconstituting normal SERCA2a levels decreases diastolic intracellular Ca²⁺ by increasing uptake into the SR and enhancing Ca²⁺ release. Beyond the contractile benefits of lowering diastolic Ca²⁺, it has been shown that sustained elevations of resting Ca²⁺ lead to activation of serine-threonine phosphatases including calcineurin inducing hypertrophy and cell death in cells (Lim (1999) Nature Medicine 5(3):246-7). Therefore a decrease in diastolic Ca²⁺ may in effect decrease the stimulation of phosphatases and reduce the pro-apoptotic and pro-hypertrophy signaling. Heart failure is associated with an increased incidence of ventricular arrhythmias and triggered activity is a probable mechanism of arrhythmogenesis in heart failure. The increase in intracellular calcium secondary to SERCA2a downregulation increases the arrhythmogenic potential. Preventing an increase in intracellular calcium by overexpression of SERCA2a prevents the induction of triggered activity. Furthermore, improvement in energetics is another important finding in these examples which may have a direct influence on survival.

Our results demonstrate that restoring SERCA2a expression can improve not only systolic and diastolic performance in failing hearts but also survival and cardiac energetics. Furthermore, SERCA2a normalization halts the adverse remodeling that occurs with congestive heart failure.

### EQUIVALENTS

Those skilled in the art will recognize, or be able to ascertain, using no more than routine experimentation, many equivalents to the specific embodiments of the invention described herein. Such equivalents are intended to be encompassed by the following claims:

1. A method of evaluating a treatment for a heart disorder, comprising:
providing a heart cell, into which has been introduced by somatic gene transfer, a nucleic acid which results in the expression of phospholamban;
administering the treatment to the heart cell; and
evaluating the effect of the treatment on the heart cell,
thereby evaluating the treatment for a heart disorder.

2. The method of claim 1, wherein the evaluation includes evaluating the effect of the treatment on a parameter related to contractility.

3. The method of claim 2, wherein the parameter related to contractility is intracellular Ca²⁺ concentration.

4. The method of claim 2, wherein the parameter related to contractility is SR Ca²⁺ ATPase activity.

5. The method of claim 1, wherein the treatment is administered in vivo.

6. The method of claim 5, wherein the treatment is administered to an experimental animal.

7. The method of claim 6, wherein the experimental animal is a transgenic animal.

8. The method of claim 7, wherein the transgenic animal expresses a transgene encoding a protein in the phospholamban pathway.

9. The method of claim 1, wherein the treatment is administered in vitro.

10. The method of claim 1, wherein the nucleic acid encodes a phospholamban protein.

11. A method of evaluating a treatment for a heart disorder, comprising:
providing a heart, into some or all the cells of which has been introduced, by somatic gene transfer, a nucleic acid which results in the expression of phospholamban;
administering the treatment to the heart; and
evaluating the effect of the treatment on the heart,
thereby evaluating the treatment for a heart disorder.

12. The method of claim 11, wherein the evaluation includes evaluating the effect of the treatment on a parameter related to contractility.

13. The method of claim 12, wherein the parameter related to contractility is intracellular Ca²⁺ concentration.

14. The method of claim 12, wherein the parameter related to contractility is SR Ca²⁺ ATPase activity.

15. The method of claim 12, wherein the parameter related to contractility is force generation.

16. The method of claim 11, wherein the treatment is administered in vivo.

17. The method of claim 11, wherein the treatment is administered to an experimental animal.

18. The method of claim 17, wherein the experimental animal is a transgenic animal.

19. The method of claim 18, wherein the transgenic animal expresses a transgene encoding a protein in the phospholamban pathway.

20. The method of claim 11, wherein the treatment is administered in vitro.

21. The method of claim 11, wherein the nucleic acid encodes a phospholamban protein.

22. A method of evaluating a treatment for a heart disorder, comprising:
providing heart tissue into some or all of the cells of which has been introduced by somatic gene transfer a nucleic acid which results in the expression of phospholamban;
administering the treatment to the heart tissue; and
evaluating the effect of the treatment on the heart tissue,
thereby evaluating the treatment for a heart disorder.

23. The method of claim 22, wherein the evaluation includes evaluating the effect of the treatment on a parameter related to contractility.

24. The method of claim 23, wherein the parameter related to contractility is intracellular Ca²⁺ concentration.

25. The method of claim 23, wherein the parameter related to contractility is SR Ca²⁺ ATPase activity.

26. The method of claim 23, wherein the parameter related to contractility is force generation.

27. The method of claim 22, wherein the treatment is administered in vivo.

28. The method of claim 22, wherein the treatment is administered to an experimental animal.

29. The method of claim 28, wherein the experimental animal is a transgenic animal.

30. The method of claim 29, wherein the transgenic animal expresses a transgene encoding a protein in the phospholamban pathway.

31. The method of claim 22, wherein the treatment is administered in vitro.

32. The method of claim 22, wherein the nucleic acid encodes a phospholamban protein.

33. A method of evaluating a treatment for a heart disorder, comprising:
providing a first and a second heart cell, into each of which has been introduced by somatic gene transfer, a nucleic acid which results in the expression of phospholamban;
administering the treatment to a first heart cell in vitro;
evaluating the effect of the in vitro treatment on the first heart cell;
administering the treatment to a second heart cell in vivo; and
evaluating the effect of the in vivo treatment on the second heart cell,
thereby evaluating the treatment for a heart disorder.

34. A method of evaluating a treatment for a heart disorder, comprising:
providing a first administration of the treatment to a heart cell, into which has been introduced by somatic gene transfer, a nucleic acid which results in the expression of phospholamban;
evaluating the effect of the first administration on the heart cell;
providing a second administration of the treatment to a heart cell, into which has been introduced by somatic gene transfer, a nucleic acid which results in the expression of phospholamban; and
evaluating the effect of the second administration on the heart cell,
thereby evaluating the treatment for a heart disorder.

35. The method of claim 34, wherein the first and second administration are administered to the same cell.

36. The method of claim 34, wherein the first and second administration are administered to different cells.

37. The method of claim 34, wherein the first and second administration are administered under the same conditions.

38. The method of claim 34, wherein the first and second administration are administered under different conditions.

39. A method of evaluating a treatment for a heart disorder, comprising:
providing a heart cell, into which has been introduced by somatic gene transfer, a nucleic acid which results in the expression of phospholamban;
administering the treatment to the heart cell;
evaluating the effect of the treatment on the heart cell;
providing a heart, into which has been introduced by somatic gene transfer, a nucleic acid which results in the expression of phospholamban;
administering the treatment to the heart; and
evaluating the effect of the treatment on the heart,
thereby evaluating the treatment for a heart disorder.

40. The method of claim 39, wherein the treatment is administered to the heart cell in vitro and to the heart in vivo.

41. The method of claim 39, wherein the treatment is administered to the heart cell and to the heart in vitro.

42. A method of delivering a compound to the heart of a subject, comprising:
restricting the aortic flow of blood out of the heart, such that blood flow is re-directed to the coronary arteries;
introducing said compound into the lumen of the circulatory system, such that said compound flows into the coronary arteries;
allowing the heart to pump while the aortic outflow of blood is restricted; and
reestablishing the flow of blood,
thereby allowing said compound to flow into and be delivered to the heart.

43. The method of claim 42, wherein the compound comprises a nucleic acid which directs the expression of a peptide.

44. The method of claim 42, wherein the compound comprises a virus vector suitable for somatic gene delivery.

45. The method of claim 40, wherein the vector is an adenovirus vector.

46. The method of claim 43, wherein the peptide is phospholamban.

47. The method of claim 42, wherein the subject is a human.

48. The method of claim 47, wherein the human is suffering from a cardiac disorder.

49. The method of claim 48, wherein the cardiac disorder is heart failure.

50. The method of claim 48, wherein the cardiac disorder is ischemia.

51. The method of claim 48, wherein the cardiac disorder is transplant rejection.

52. The method of claim 42, wherein the subject is an experimental animal.

53. The method of claim 52, wherein the experimental animal is a transgenic animal.

54. The method of claim 53, wherein the transgenic animal expresses a transgene encoding a protein in the phospholamban pathway.

55. The method of claim 42, wherein the blood flow restriction is achieved by obstructing the aorta.

56. The method of claim 55, further comprising obstructing the pulmonary artery.

57. The method of claim 42, further comprising opening the pericardium.

58. The method of claim 42, wherein the compound is introduced by a catheter.

59. The method of claim 43, wherein the nucleic acid, which directs the expression of the peptide, is homogeneously overexpressed in the heart of the subject.

60. The method of claim 42, wherein the compound is introduced into the aortic root.

61. The method of claim 42, wherein the compound is introduced into the lumen of the heart.

62. A heart cell, into which has been introduced by somatic gene transfer, a nucleic acid which results in the expression of phospholamban.

63. A heart tissue, into which has been introduced by somatic gene transfer, a nucleic acid which results in the expression of phospholamban.

64. A heart, into which has been introduced by somatic gene transfer, a nucleic acid which results in the expression of phospholamban.

65. A method of treating a subject, comprising:
introducing into a heart cell a nucleic acid which results in the expression of SERCA2a,
thereby treating the subject.

66. The method of claim 65, wherein treating the subject comprises modulating the ratio of phospholamban to SERCA2a in a heart cell of the subject.

67. The method of claim 65, wherein the subject is at risk for, or has, a heart disorder.

68. The method of claim 67, wherein the heart disorder is heart failure, ischemia, arrhythmia, myocardial infarction, congestive heart failure, transplant rejection, abnormal heart contractility, or abnormal Ca+2 metabolism.

69. The method of claim 65, wherein the nucleic acid is introduced by somatic gene transfer.

70. The method of claim 65, wherein the subject is a human.

71. The method of claim 69, wherein the nucleic acid is introduced in vitro.

72. The method of claim 69, wherein the nucleic acid is introduced in vivo.

73. A method of treating a subject, comprising:
introducing into the subject a nucleic acid which results in the expression of an antisense nucleic acid which is at least partially complementary to a phospholamban DNA sequence.

74. The method of claim 73, wherein the subject is at risk for, or has, a heart disorder.

75. The method of claim 73, wherein the heart disorder is heart failure, ischemia, arrhythmia, myocardial infarction, congestive heart failure, transplant rejection, abnormal heart contractility, or abnormal Ca+2 metabolism.

76. The method of claim 73, wherein the nucleic acid is introduced by somatic gene transfer.

77. The method of claim 73, wherein the subject is a human.

78. The method of claim 76, wherein the nucleic acid is introduced in vitro.

79. The method of claim 76, wherein the nucleic acid is introduced in vivo.

80. A method of treating a heart cell of a subject, comprising:
introducing into the subject a first nucleic acid which results in the expression of an antisense nucleic acid which is at least partially complementary to a phospholamban DNA sequence; and
introducing into the subject a second nucleic acid which results in the expression of SERCA2,
thereby treating a heart cell of a subject.

81. The method of claim 80, wherein the subject is at risk for, or has, a heart disorder.

82. The method of claim 80, wherein the heart disorder is heart failure, ischemia, arrhythmia, myocardial infarction, congestive heart failure, transplant rejection, abnormal heart contractility, or abnormal Ca+2 metabolism.

83. The method of claim 80, wherein the nucleic acid is introduced by somatic gene transfer.

84. The method of claim 80, wherein the subject is a human.

85. The method of claim 80, wherein the nucleic acid is introduced in vitro.

86. The method of claim 80, wherein the nucleic acid is introduced in vivo.
